(19) United States
(12) Patent Application Publication
(10) Pub. No.: US 2011/0129489 A1
(43) Pub. Date: Jun. 2, 2011

(54) METHODS FOR GENERATING AN IMMUNE RESPONSE USING DNA AND A VIRAL VECTOR

(76) Inventors: Erik Depla, Destelbergen (BE); Annegret Van Der Aa, Berendrecht (BE); Sofie De Schepper, Strombeek-bever (BE); Stany Depraetere, Oudenaarde (BE); Karen De Vreese, Zingem (BE)

(21) Appl. No.: 12/452,494

(22) PCT Filed: Jul. 7, 2008
(86) PCT No.: PCT/EP2008/058759

§ 371 Date: Nov. 5, 2010

Related U.S. Application Data
(60) Provisional application No. 60/929,708, filed on Jul. 10, 2007.

(30) Foreign Application Priority Data
Jul. 6, 2007 (EP) 07111980.4

(51) Int. Cl. A61K 39/29; A61K 39/275; A61P 31/20; A61P 31/14; A61P 37/04

(52) U.S. Cl. 424/189.1; 424/232.1; 424/227.1; 424/228.1; 424/208.1

(57) ABSTRACT

The present invention relates to the generation of an immune response against a target antigen using a DNA and viral vector in a specific administration pattern.

### FIELD OF THE INVENTION

The present invention relates to the generation of an immune response against a target antigen using DNA and a viral vector in a specific administration pattern.

### BACKGROUND ART

Vaccines for infectious diseases represent an important field of current research. The lack of effective vaccination schemes for these complex diseases represents a major obstacle in the generation of an antigen-specific immune response.

The main bottleneck in developing vaccines for intracellular infections such as HBV, HCV, HPV, HIV, and malaria is the ability to induce strong and long-lasting cell mediated immunity. Stimulation of a functional CD8+ response is often crucial in addition to a Th1-type CD4+ T-cell response. The use of recombinant viral vectors is more and more popular in order to achieve intracellular antigen expression that can result in epitope presentation on MHC class I molecules thus allowing the induction of pathogenic CD8+ T-cell responses.

Based on data shown in literature (McConkey et al 2003, Mwau et al. 2004, Vuola et al 2005) heterologous prime-boost vaccination regimens that combine two different vectors encoding the same antigen is more efficient in inducing cell mediated immune response than the use of a single vector. A variety of combinations of prime and boost have been tested in different potential vaccine regimes. Nevertheless, said regimens were thus far not successful in consistently inducing cellular responses in humans.

In view of the heterogeneous immune response observed with viral infection, induction of a multi-specific cellular immune response directed simultaneously against multiple epitopes is important for the development of an efficacious vaccine.

The technology relevant to polyepitope vaccines is developing and a number of different approaches are available which allow simultaneous delivery of multiple epitopes. Several independent studies have established that induction of simultaneous immune responses against multiple epitopes can be achieved. In terms of immunization with polyepitope nucleic acid vaccines, several examples have been reported where multiple T-cell responses were induced. Specifically, minigene vaccines composed of a plurality of epitopes have been shown to be active (Woodberry et al., 1999, Thomson et al., 1998, Mateo et al., 1999, Ishioka et al., 1999, WO04/031210 (Pharmexa Inc. et al.), WO05/089164 (Pharmexa Inc. et al.) and WO01/21189 (Pharmexa Inc.)).

A major problem however has been the identification of a means of inducing a sufficiently strong immune response in a subject to protect against infection and disease. So, although many antigens are known that might be useful in treating infectious disease the problem has been how to deliver such antigens in a way that induces a sufficiently strong immune response of a particular type.

Accordingly, effective schemes for administration of vaccine protocols are needed. Therefore, it is an object of the present invention to develop a novel immunization scheme for inducing a strong immune response. Said immunization scheme is especially useful for generating high levels of cytotoxic T lymphocytes (CTL) and/or T Helper Lymphocytes (HTL). It is another object of the invention to provide a heterologous prime boost regimen for the prevention or treatment of disease, specifically infectious disease.

### SUMMARY OF THE INVENTION

The present invention relates to the generation of an immune response against a target antigen using DNA and a viral vector in a specific administration pattern.

In a first embodiment, the invention encompasses a method for preventing and/or treating an infection comprising at least two cycles of DNA-viral vector administration.

More specific, the invention relates to the use of a DNA and viral vector encoding an antigen derived from a pathogen in the manufacture of a medicament for preventing and/or treating an infection, wherein the administration pattern of the medicament comprises at least two cycles of DNA-viral vector administration,

wherein DNA is a plasmid DNA encoding said antigen, and
wherein the viral vector is a non replicating or replication impaired recombinant poxvirus which directs the expression of said antigen.

Even more specific, the invention relates to the use of a DNA and viral vector encoding an antigen derived from a pathogen in the manufacture of a medicament for preventing and/or treating an infection, wherein the medicament is prepared for administration of at least two cycles of DNA-viral vector, wherein DNA is a plasmid DNA encoding said antigen, and wherein the viral vector is a non replicating or replication impaired recombinant poxvirus which directs the expression of said antigen.

In a further embodiment, the DNA and viral vector are administered with an interval of one to twelve weeks between the DNA and viral vector administration within one cycle, and with an interval of 1 week to 1 year between two subsequent cycles.

In a particular embodiment, the DNA is administered intramuscular, and the viral vector is administered subcutaneous, intradermal or intramuscular. DNA delivery can be via electroporation, via facilitated delivery, via cationic lipid complexes, via particle-mediated or via pressure-mediated delivery. Specific dosages are between 1 μg and 5 mg for DNA and between 1×10E5 and 5×10E9 pfu for the viral vector.

In a further embodiment of the invention, the administration pattern of the medicament comprises at least the following:

a) n₁ DNA—b) m₁ viral vector—c) n₂ DNA—d) m₂ viral vector, wherein n₁ and/or n₂ equals 1 to 5 times administration of DNA, and wherein m₁ and/or m₂ equals 1 to 5 times administration of the viral vector. More specific, the interval within steps (a), (b), (c) and/or (d) is one to twelve weeks if n₁, m₁, n₂, and/or m₂ is greater than 1. Typically, the interval between step (a) and (b), and between step (c) and (d) is one to twelve weeks, and the interval between step (c) and (d) is 1 week to 1 year.

In a particular embodiment, the medicament is administered at one to four weeks interval within and/or between the steps (a), (b), (c), and (d).

In a particular embodiment, the non replicating or replication impaired recombinant poxvirus is a vaccinia virus, more specific MVA. Furthermore, the pathogen is a virus, the antigen is a viral antigen and the infection is a viral infection.

In a more specific embodiment, the viral antigen is obtained from HBV, HCV, HIV or HPV and the viral infection is a HCV, HBV, HIV, or HPV infection.

The antigen as described in the present invention is a protein, an immunogenic portion thereof, or a combination of proteins and/or immunogenic portions. In a particular embodiment, the antigen is a polyepitope construct. Preferably, the polyepitope construct comprises at least 10 CTL epitopes. More specific, the polyepitope construct comprises at least two of the CTL epitopes selected from the group consisting of SEQ ID NO 1-30. Optionally, the polyepitope construct further comprises at least one HTL epitope. More particular, at least one HTL epitope is selected from the group consisting of: SEQ ID NO 31-47. Even more particular, the polyepitope construct is characterized by SEQ ID NO 49.

In a further embodiment, the antigen encoded by the DNA and viral vector used in the prime boost regimen of the present invention is a polyepitope construct comprising the following CTL epitopes: SEQ ID NO 1-30. More specific, the polyepitope construct further comprises the following HTL epitopes: SEQ ID NO 31-47.

In a particular embodiment, the administration pattern comprises the following:

DNA—3 weeks—DNA—3 weeks—MVA—3 weeks—DNA-3 weeks—MVA, whereby
the DNA dosage is 4 mg for intramuscular injection; and
the MVA dosage is 2×10E8 pfu for subcutaneous injection.

The present invention also envisages a kit for preventing and/or treating an infection, comprising:

a) a DNA priming composition encoding an antigen derived from a pathogen; and
b) a viral vector boosting composition which directs the expression of said antigen,
wherein the viral vector is a non replicating or replication impaired recombinant poxvirus.

In a specific embodiment, the kit further comprises instructions for administration comprising an administration pattern of at least two cycles of DNA-viral vector.

### FIGURE LEGENDS

FIG. 1: Amino acid sequence of the construct INX102-3697 (SEQ ID NO 49).

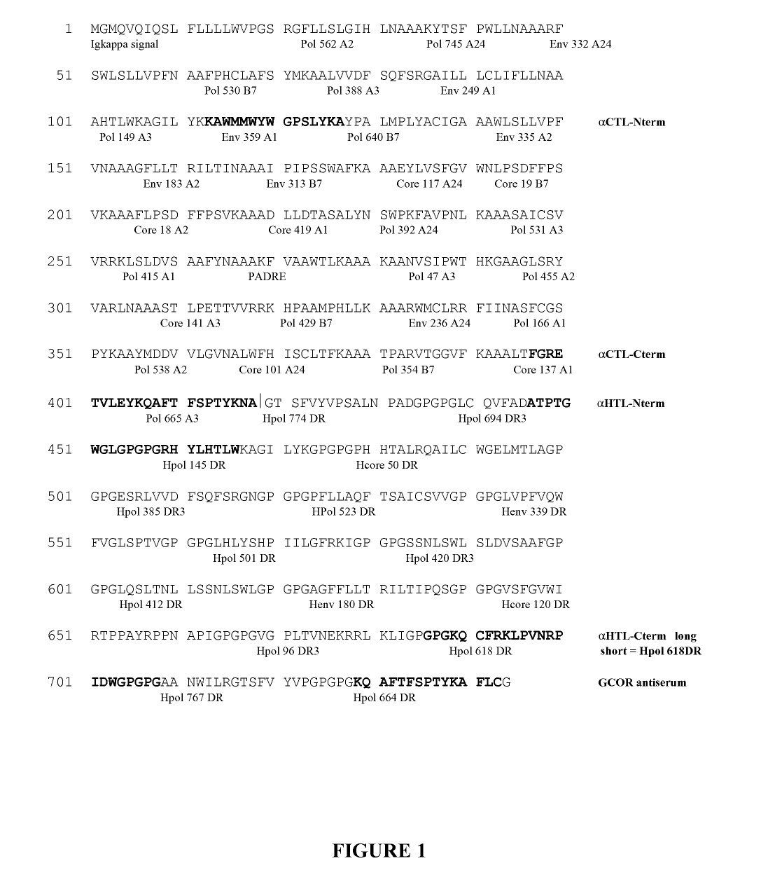

FIG. 2: Schematic diagram of the HBV DNA construct INX102-3697. The orientation of the CTL and HTL epitopes in the synthetic gene is shown in the upper part, the HLA restriction of each epitope, with respect to supertype, is also shown. The functional elements of the DNA plasmid vector are indicated in the lower part of the figure.

FIG. 3: Median cumulative CTL responses in HLA-A02/Kb transgenic mice.

FIG. 4: Median cumulative HTL responses in HLA-A02/Kb transgenic mice.

### DETAILED DESCRIPTION OF THE INVENTION

Unless defined otherwise, all technical and scientific terms used herein have the same meaning as commonly understood by one of ordinary skill in the art. All publications mentioned herein are incorporated by reference.

The present invention relates to methods of vaccination for the effective generation of an antigen-specific immune response in a mammal, preferably a human. Specifically, the present invention relates to heterologous prime boost immunization regimens for the generation of a cellular and/or humoral immune response, and more specific a Cytotoxic T Lymphocyte (CTL), a T Helper Lymphocyte (HTL) response and/or an antibody response.

The method of the present invention is effective in treating or preventing disease. Many diseases have specific antigens associated with the disease state. Such antigens or epitopes of these antigens are crucial to immune recognition and ultimate elimination or control of the disease in a patient. The invention provides a vaccine approach based on a heterologous prime boost regimen. Specifically, said regimen includes the administration of a DNA and viral vector encoding an antigen in repeated cycles of DNA-viral vector. It has been demonstrated that said treatment regimen results in a very broad and vigorous immune response.

In a first embodiment, the present invention envisages the use of a DNA and viral vector encoding an antigen derived from a pathogen in the manufacture of a medicament for preventing and/or treating an infection, wherein the administration pattern of the medicament comprises at least two cycles of DNA—viral vector administration.

The “medicament” is comprised of at least one DNA vector and at least one viral vector, encoding for the same antigen. The DNA or viral vector is also generally referred to as “vector”.

As used herein, the term “antigen” relates to a complete protein derived from a pathogen, or an immunogenic portion thereof. Also combinations of proteins, combinations of one or more proteins and one or more immunogenic portions, and combinations of immunogenic portions are included. The term “immunogenic” or “immunogenicity” as used herein is the ability to evoke an immune response, i.e. a humoral and/or cellular response. The term “humoral immune response” refers to an immune response mediated by antibody molecules secreted by B-lymphocytes, while a “cellular immune response” is one mediated by T-lymphocytes and/or other white blood cells.

An “immunogenic portion” refers to a fragment of a protein that evokes an immune response and contains one or more epitopes. The immunogenic portion may be of varying length, although it is generally preferred that the portion is at least 7 amino acids long up to the full length protein. In a specific embodiment, the immunogenic portion is a T-cell epitope or a B-cell epitope. Immunogenicity can be manifested in several different ways. Immunogenicity corresponds to whether an immune response is elicited at all, and to the vigor of any particular response, as well as to the extent of a population in which a response is elicited. For example, a peptide might elicit an immune response in a diverse array of the population, yet in no instance produce a vigorous response.

Particularly preferred immunogenic portions may be determined by a variety of methods. For example, identification of immunogenic portions of the protein may be predicted based upon amino acid sequence. Briefly, various computer programs which are known to those of ordinary skill in the art may be utilized to predict CTL and HTL epitopes. Other assays, however, may also be utilized, including, for example, ELISA which detects the presence of antibodies against the antigen, as well as assays which test for CTL and/or HTL epitopes, such as ELISPOT and proliferation assays.

Various strategies can be utilized to evaluate T-cell immunogenicity, including but not limited to:

1) Evaluation of primary T-cell cultures from normal individuals (see, e.g., Wentworth et al., 1995; Celis et al., 1994; Tsai et al., 1997; Kawashima et al., 1998). This procedure involves the stimulation of peripheral blood lymphocytes (PBL) from normal subjects with a test peptide in the presence of antigen-presenting cells in vitro over a period of several weeks. T-cells specific for the peptide become activated during this time and are detected using, e.g., a ⁵¹Cr-release assay involving peptide sensitized target cells.
2) Immunization of HLA transgenic mice (see, e.g., Wentworth et al., 1996; Alexander et al., 1997) or mice having MHC that resembles HLA. In this method, peptides (e.g. formulated in incomplete Freund's adjuvant) are administered subcutaneously to HLA transgenic mice or surrogate mice. Eleven to 14 days following immunization, splenocytes are removed. Cells are cultured in vitro in the presence of test peptide for approximately one week and peptide-specific T-cells are detected using, e.g., a ⁵¹Cr-release assay involving peptide-sensitized target cells and/or target cells expressing endogenously generated antigen. Alternatively, cells are incubated overnight together with peptide-loaded APC in the IFNg ELISPOT assay for the quantitation of peptide-specific single T-cells releasing mouse interferon gamma upon stimulation.
3) Demonstration of recall T-cell responses from immune individuals who have effectively been vaccinated, recovered from infection, and/or from chronically infected patients (see, e.g., Rehermann et al., 1995; Doolan et al., 1997; Bertoni et al., 1997; Threlkeld et al., 1997; Diepolder et al., 1997). In applying this strategy, recall responses are detected by culturing PBL from subjects that have been naturally exposed to the antigen, for instance through infection, and thus have generated an immune response “naturally”, or from patients who were vaccinated with a vaccine comprising the epitope of interest. PBL from subjects are cultured in vitro up to 2 weeks in the presence of test peptide plus antigen presenting cells (APC) to allow activation of “memory” T-cells, as compared to “naive” T-cells. At the end of the culture period, T-cell activity is detected using assays including ⁵¹Cr release involving peptide-sensitized target cells, T-cell proliferation, or cytokine release.

The medicament as described herein can have a therapeutic use or a prophylactic use. The therapeutic use refers to a medicament aimed for treatment of infection and to be administered to patients being infected. The prophylactic use refers to a medicament aimed for preventing infection and to be administered to healthy persons who are not yet infected.

As used herein, the term “pathogen” relates to any agent capable of causing disease. The term “infection” includes bacterial, protozoan, yeast or viral infection. In a specific embodiment, the infection is an intracellular infection. Such infection is caused by intracellular pathogens including but not limited to mycobacteria, Chlamydia, Legionella, malaria parasites, Aspergillus, Candida, poxviruses, the hepatitis C virus (HCV), the hepatitis B virus (HBV), the human papilloma virus (HPV), the Human Immunodeficiency virus (HIV), influenza, Epstein-Barr virus (EBV), cytomegalovirus (CMV), members of the (human) herpes virus family, measles, dengue and HTLV.

Representative examples of proteins as suitable antigens used in the prevention or treatment of disease are described in the literature and well known to the skilled person. For example, mycobacterial antigens include Mycobacteria tuberculosis proteins from the fibronectin-binding antigen complex (Ag 85). Examples of suitable malaria parasite antigens include the circumsporozoite protein of Plasmodium falciparum. For HIV, particularly preferred antigens include the HIV gag and env proteins (gp-120, p17, gp-160 antigens). The hepatitis B virus presents several different antigens including among others, three HB “Surface” antigens (HBsAgs), an HBcore antigen (HBcAg), an HB e-antigen (HBeAg), and an HB x-antigen (HBxAg). Also presented by HBV are polymerase (“HBV pol”), ORF 5, and ORF 6 antigens. Preferred immunogenic portion(s) of hepatitis C(HCV) may be found in the UTR, Core, E1, E2 and NS3-NS5 regions. For HPV, immunogenic portions are present in or represented by the L1, L2, E1, E2, E4, E5, E6 and E7 proteins.

According to a specific embodiment of the invention, the pathogen is a virus, the infection is a viral infection and the antigen is a viral protein or an immunogenic portion thereof. More particular, the viral antigen is obtained from HBV, HCV, HPV or HIV and the viral infection is a HBV, HCV, HPV or HIV infection.

As will be evident to one of ordinary skill in the art, various immunogenic portions of the herein described proteins may be combined in order to present an immune response when administered by one of the vectors of the present invention. In a specific embodiment, the antigen is a polyepitope construct comprising a combination of at least two immunogenic portions. The term “construct” as used herein generally denotes a composition that does not occur in nature. As such, the “polyepitope construct” of the present invention does not encompass a wild-type full-length protein but includes a chimeric protein containing isolated epitopes from at least one protein, not necessarily in the same sequential order as in nature. Said epitopes are “isolated” or “biologically pure”. The term “isolated” refers to material that is substantially free from components that normally accompany it as found in its naturally occurring environment. However, it should be clear that the isolated epitope of the present invention might comprise heterologous cell components or a label and the like. An “isolated” epitope refers to an epitope that does not include the neighbouring amino acids of the whole sequence of the antigen or protein from which the epitope was derived.

With regard to a particular amino acid sequence, an “epitope” is a set of amino acid residues which is involved in recognition by a particular immunoglobulin, or in the context of T-cells, those residues necessary for recognition by T-cell receptor proteins and/or Major Histocompatibility Complex (MHC) molecules.

The term “peptide” designates a series of amino acids, connected one to the other, typically by peptide bonds between the amino and carboxyl groups of adjacent amino acids.

The epitopes are of a certain length and bind to a molecule functioning in the immune system, preferably a HLA class I, HLA class II and a T-cell receptor. The epitopes in a polyepitope construct can be HLA class I epitopes and/or HLA class II epitopes. HLA class I epitopes are referred to as CTL epitopes and HLA class II epitopes are referred to as HTL epitopes. Some polyepitope constructs can have a subset of HLA class I epitopes and another subset of HLA class II epitopes. A CTL epitope usually consists of 13 or less amino acid residues in length, 12 or less amino acids in length, or 11 or less amino acids in length, preferably from 8 to 13 amino acids in length, most preferably from 8 to 11 amino acids in length (i.e. 8, 9, 10, or 11). A HTL epitope consists of 50 or less amino acid residues in length, and usually from 6 to 30 residues, more usually from 12 to 25, and preferably consists of 15 to 20 (i.e. 15, 16, 17, 18, 19, or 20) amino acids in length.

In a particular embodiment, the DNA and viral vector encoding an antigen is used to induce a cellular immune response. More specific, the antigen induces a CTL response. The term “Cytotoxic T Lymphocyte (CTL) response” as used herein refers to a specific cellular immune response mediated by CD8+ cells. This specific cellular immune response can be e.g. the production of specific cytokines such as IFN-gamma (measured e.g. by ELISPOT or intracellular FACS), degranulation (measured e.g. by a granzyme-b specific ELISPOT), or cytolytic activity (e.g. measured by a ⁵¹Cr-release assay). Alternatively the antigen specific CD8+ cell can be detected directly by e.g. the use of tetramers.

CTL epitopes have been identified and can be found in literature for many different diseases. It is the aim of the present invention to provide a method of immunising against diseases in which CTL responses play a protective role.

The polyepitope construct of the present invention preferably comprises 2 or more, 5 or more, 10 or more, 13 or more, 15 or more, 20 or more, or 25 or more CTL epitopes. More specific, the polyepitope construct comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60 or more CTL epitopes. In a further embodiment, the polyepitope construct of the invention further comprises one or more HTL (T Helper) epitopes. At least one HTL epitope can be derived from any target antigen. As such, the polyepitope construct of the present invention optionally comprises at least 1, 2, 3, 4, 5, 6, 7, 8, 9, 10, 11, 12, 13, 14, 15, 16, 17, 18, 19, 20, 25, 30, or more HTL epitopes. In a preferred embodiment, the polyepitope construct of the present invention comprises the universal T-cell epitope called PADRE® (Pharmexa Inc.; described, for example in U.S. Pat. No. 5,736,142 or International Application WO95/07707, which are enclosed herein by reference). A “PanDR binding peptide or PADRE® peptide” is a member of a family of molecules that binds more that one HLA class II DR molecule. The pattern that defines the PADRE® family of molecules can be thought of as an HLA class II supermotif. PADRE® binds to most HLA-DR molecules and stimulates in vitro and in vivo human helper T lymphocyte (HTL) responses.

In another embodiment, the polyepitope construct of the present invention comprises one or more HTL epitopes derived from the same pathogen as the CTL epitopes.

Alternatively HTL epitopes can be used from universally used vaccines such as tetanos toxoid. It may also be useful to include B cell epitopes in the polyepitope construct for stimulating B cell responses and antibody production.

In a specific embodiment, the invention is directed to the use of a polyepitope construct in the manufacture of a medicament for preventing and/or treating an infection by administering the medicament according to the treatment regimen as described herein. More specific, the polyepitope construct contains 2, 3, 4, 5, 10, 15, or more epitopes derived from a virus. More particular, two or more CTL epitopes in the polyepitope construct are derived from the Hepatitis B virus (HBV), and more specifically from the HBV Core protein, the HBV Polymerase protein and/or the HBV Envelope protein. Even more particular, two or more CTL epitopes are selected from the list of epitopes given in Table 1. In a preferred embodiment, the polyepitope construct as described herein comprises all the CTL epitopes given in Table 1. Optionally, the polyepitope construct furthermore comprises one or more HTL epitopes. More particular, at least one HTL epitope is selected from the list of epitopes given in Table 2. In a preferred embodiment, the polyepitope construct as described herein comprises all the HTL epitopes given in Table 2.

The epitopes of the polyepitope construct are directly or indirectly linked to one another. More specific, two or more of the epitopes (either CTL and/or HTL) are either contiguous or are separated by a linker or one or more spacer amino acids. “Link” or “join” refers to any method known in the art for functionally connecting epitopes. More particular, the polyepitope construct of the present invention is a recombinant string of two or more epitopes.

In a specific embodiment, the polyepitope construct of the present invention further comprises one or a plurality of spacer amino acids between two or more epitopes. More specific, the polyepitope construct comprises 1 to 9, and more preferably 1 to 5 spacer amino acids, i.e. 1, 2, 3, 4 or 5 spacer amino acids between two or more, or all, of the epitopes in the construct. A “spacer” refers to a sequence that is inserted between two epitopes in a polyepitope construct to prevent the occurrence of junctional epitopes (an epitope recognized by the immune system, not present in the target antigen, and only created by the man-made juxtaposition of epitopes), or to facilitate cleavage between epitopes and thereby enhance epitope presentation.

To develop polyepitope constructs using the epitopes of the present invention, said epitopes can be sorted and optimized using a computer program or, for fewer epitopes, not using a computer program. “Sorting epitopes” refers to determining or designing an order of the epitopes in a polyepitope construct.

“Optimizing” refers to increasing the antigenicity of a polyepitope construct having at least one epitope pair by sorting epitopes to minimize the occurrence of junctional epitopes, and inserting a spacer residue (as described herein) to further prevent the occurrence of junctional epitopes or to provide a flanking residue. As described herein, a “flanking residue” is a residue that is positioned next to an epitope. A flanking residue can be introduced or inserted at a position adjacent to the N-terminus (N+1) or the C-terminus (C+1) of an epitope. An increase in immunogenicity or antigenicity of an optimized polyepitope construct is measured relative to a polyepitope construct that has not been constructed based on the optimization parameters by using assays known to those skilled in the art, e.g. assessment of immunogenicity in HLA transgenic mice, ELISPOT, tetramer staining, ⁵¹Cr release assays, and presentation on antigen presenting cells in the context of MHC molecules. The process of optimizing polyepitope constructs is given e.g. in WO01/47541 and WO04/031210 (Pharmexa Inc. et al.; incorporated herein by reference). It is preferred that spacers are selected by concomitantly optimizing epitope processing and preventing junctional motifs.

The “spacer amino acid” or “spacer peptide” is typically comprised of one or more relatively small, neutral molecules, such as amino acids or amino acid mimetics, which are substantially uncharged under physiological conditions. For example, spacers flanking HLA class II epitopes preferably include G (Gly), P (Pro), and/or N (Asn) residues. A particularly preferred spacer for flanking a HLA class II epitope includes alternating G and P residues, for example, (GP)n, (PG)n, (GP)nG, (PG)nP, and so forth, where n is an integer between 1 and 10, preferably 2 or 3, and where a specific example of such a spacer is GPGPG (SEQ ID NO 48). For separating class I epitopes, or separating a class I and a class II epitope, the spacers are typically selected from, e.g., A (Ala), N (Asn), K (Lys), G (Gly), L (Leu), I (Ile), R (Arg), Q (Gln), S (Ser), C (Cys), P (Pro), T (Thr), or other neutral spacers of nonpolar amino acids or neutral polar amino acids, though polar residues could also be present. A preferred spacer, particularly for HLA class I epitopes, comprises 1, 2, 3 or more consecutive alanine (A), lysine (K) or asparagine (N) residues, or a combination of K (Lys) and A (Ala) residues, e.g. KA, KAA or KAAA, or a combination of N (Asn) and A (Ala) residues, e.g. NA, NAA or NAAA, or a combination of G (Gly) and A (Ala) residues, e.g. GA or GAA. The present invention is thus directed to a polypeptide comprising a polyepitope construct as described herein, and wherein the epitopes in the construct are separated by one or more spacer amino acids. In a preferred embodiment, the one or more spacer amino acids are selected from the group consisting of: K, R, N, Q, G, A, S, C, G, P and T.

In a specific embodiment, the antigen of the present invention is the construct represented by SEQ ID NO 49. Characteristics of the construct (GCR-3697) have been described in WO04/031210 (Pharmexa Inc.; incorporated herein by reference).

According to the present invention, the DNA and viral vector encoding the antigen is administered to the subject (being a mammal, preferably a human) using a specific immunization protocol. Said protocol includes a repeated cycle of subsequent DNA and viral vector administrations. In a specific embodiment, the invention relates to the use of DNA and a viral vector encoding an antigen derived from a pathogen in the manufacture of a medicament for preventing and/or treating an infection, wherein the administration pattern of the medicament comprises at least two cycles of DNA-viral vector administration, wherein said DNA is a plasmid DNA (pDNA) encoding said antigen, and wherein the viral vector is a non replicating or replication impaired recombinant poxvirus which directs the expression of said antigen.

The DNA or viral vector is also generally referred to as “vector”.

As used herein, the term “viral vector” relates to non replicating or replication impaired recombinant poxvirus which directs the expression of said antigen.

The term “non-replicating” or “replication-impaired” as used herein means not capable of replication to any significant extent in the majority of normal mammalian cells or normal human cells. Viruses which are non-replicating or replication-impaired may have become so naturally (i.e. they may be isolated as such from nature) or artificially e.g. by breeding in vitro or by genetic manipulation, for example deletion of a gene which is critical for replication. There will generally be one or a few cell types of non-human origin in which the viruses can be grown, such as CEF cells for MVA. Replication of a virus is generally measured in two ways: 1) DNA synthesis and 2) viral titer.

More precisely, the term “non-replicating or replication-impaired” as used herein and as it applies to poxviruses means viruses which satisfy either or both of the following criteria: 1) exhibit a 1 log(10 fold) reduction in DNA synthesis compared to the Copenhagen strain of vaccinia virus in MRC-5 cells (a human cell line); 2) exhibit a 2 log reduction in viral titer in HELA cells (a human cell line) compared to the Copenhagen strain of vaccinia virus. Examples of poxviruses which fall within this definition are MVA, NYVAC and avipox viruses.

It will be evident that vaccinia virus strains derived from MVA, or independently developed strains having the features of MVA which make MVA particularly suitable for use in a vaccine, will also be suitable for use in the invention. As an example of this approach, MVA is used as a vector to express nucleotide sequences that encode the antigen of the invention. Upon introduction into a host, the recombinant vaccinia virus expresses the antigen or immunogenic peptide, and thereby elicits an immune response. Vaccinia vectors, for example Modified Vaccinia Ankara (MVA), and methods useful in immunization protocols are described in, e.g., U.S. Pat. No. 4,722,848.

In a particular embodiment, the vector of the present invention further comprises one or more regulatory sequences. By “regulatory sequence” is meant a polynucleotide sequence that contributes to or is necessary for the expression of an operably associated nucleic acid or nucleic acid construct in a particular host organism. The regulatory sequences that are suitable for eukaryotes, for example, include a promoter (e.g. CMV promoter), optionally an enhancer sequence, introns with functional splice donor and acceptor sites, a Kozak consensus sequence, signal sequences (e.g. Ig kappa light chain signal sequence), an internal ribosome entry site (IRES), and polyadenylation signals (e.g. SV40 early poly-A signal). Other specific examples of regulatory sequences are described herein and otherwise known in the art. A typical expression cassette thus contains all necessary regulatory elements required for efficient transcription and translation of the gene.

Suitable promoters are well known in the art and described, e.g., in Sambrook et al. (1989) and in Ausubel et al. (1994). Eukaryotic expression systems for mammalian cells are well known in the art and are commercially available. Such promoter elements include, for example, cytomegalovirus (CMV), Rous sarcoma virus long terminal repeats (RSV LTR) and Simian Virus 40 (SV40). See, e.g., U.S. Pat. Nos. 5,580,859, 5,589,466 and 5,017,487 for other suitable promoter sequences.

In addition to a promoter sequence, the expression cassette can also contain a transcription termination region downstream of the structural gene to provide for efficient termination. The termination region may be obtained from the same gene as the promoter sequence or may be obtained from different genes.

Additional vector modifications may be desired to optimize epitope expression and immunogenicity. In some cases, introns are required for efficient gene expression, and one or more synthetic or naturally-occurring introns could be incorporated into the transcribed region. The inclusion of mRNA stabilization sequences and sequences for replication in mammalian cells may also be considered for increasing antigen expression. In addition, immunostimulatory sequences (ISSs or CpGs) appear to play a role in the immunogenicity of nucleic acid vaccines. These sequences may be included in the vector, outside the polynucleotide coding sequence, if desired to enhance immunogenicity. In some embodiments, a bi-cistronic expression vector which allows production of both the antigen and a second protein (included to enhance or decrease immunogenicity) can be used. Examples of proteins or peptides that could beneficially enhance the immune response if co-expressed include cytokines (e.g., IL-2, IL-12, GM-CSF), cytokine-inducing molecules (e.g., LeIF), costimulatory molecules, or for HTL responses, pan-DR binding peptides (PADRE®, Epimmune, San Diego, Calif.).

Helper (HTL) epitopes can be joined to intracellular targeting signals and expressed separately from expressed CTL epitopes; this allows direction of the HTL epitopes to a cell compartment different than that of the CTL epitopes. If required, this could facilitate more efficient entry of HTL epitopes into the HLA class II pathway, thereby improving HTL induction. In contrast to HTL or CTL induction, specifically decreasing the immune response by co-expression of immunosuppressive molecules (e.g. TGF-β) may be beneficial in certain diseases.

The DNA and viral vector encoding the antigen of the present invention are used in a heterologous prime boost regimen. The term “heterologous” as used herein refers to a different presentation format (or vector) of the antigen, i.e. DNA or viral vector, in the priming versus the boosting agent. It is to be understood that the term “prime boost regimen” or “prime boost treatment regimen” refers to the administration of the compounds in a certain order and with a certain time interval. A “prime boost regimen” or “prime boost treatment regimen” can consist of one or multiple, i.e. two, three four or more, prime boost cycles. The term “prime” or “priming” as used herein refers to the composition administered first in a prime boost cycle. The term “boost” or “boosting” as used herein refers to the composition administered, in a prime boost cycle, with a certain time interval after the prime or priming. Specifically, the “DNA-viral vector” cycle of the present invention comprises plasmid DNA (pDNA) as priming vector and a viral vector as a boosting vector. It is to be understood that the prime may consist of more than one administration (separated in time and/or site of injection) of the same vector or composition. It is also to be understood that the boost may consist of more than one administration (separated in time and/or site of injection) of the same vector or composition. In its broadest interpretation, the time interval between prime and boost in one cycle or between two cycles can go from one day to 24 weeks or even up to 1 year. More specific, the time interval between the administrations of DNA and viral vector within 1 cycle includes 1 week, 2 weeks, 3 weeks, 4 weeks, 5 weeks, 6 weeks, 7 weeks, 8 weeks, 9 weeks, 10 weeks, 11 weeks or 12 weeks and any time interval in between. Similarly, the time interval between two cycles of “DNA-viral vector” includes 1 week, 2 weeks, 3 weeks, 6 weeks, 8 weeks, 10 weeks, 15 weeks, 20 weeks, 30 weeks, 40 weeks, and up to one year. Specifically, the time interval between the administrations of DNA and viral vector is from 1 to 12 weeks. More specific, the time interval is 1 to 4 weeks. Even more specific, the time interval is 2 or 3 weeks, plus or minus 1 to 3 days. Due to specific circumstances (e.g. illness), it may not be possible to stick at exact 2 or 3 weeks interval, therefore a deviation of 1 to 3 days is permissible.

It has been demonstrated by the present invention that in order to induce a broader and more vigorous immune response, at least two “DNA prime-viral vector boost” cycles are required as part of an immunization scheme or vaccination schedule. In the method of the present invention, the heterologous prime boost or “DNA—viral vector cycle” is repeated at least twice, and optionally three, four or five times. Accordingly, the present invention relates to the use of a DNA and viral vector encoding an antigen derived from a pathogen in the manufacture of a medicament for preventing and/or treating an infection, wherein the administration pattern of the medicament comprises at least two cycles of DNA—viral vector administration. Furthermore, the present invention relates to a medicament comprising

a) a DNA priming composition encoding an antigen derived from a pathogen; and
b) a viral vector boosting composition which directs the expression of said antigen,
wherein the viral vector is a non replicating or replication impaired recombinant poxvirus,
for use in preventing and/or treating an infection by at least two cycles of DNA-viral vector administration.

As used herein, the phrase “two cycles of DNA-viral vector administration” relates to a repeated and successive administration of the cycle “DNA prime—viral vector boost”. Accordingly, the administration pattern of the medicament comprises at least the following:

a) n₁ DNA—b) m₁ viral vector—c) n₂ DNA—d) m₂ viral vector, wherein n₁ and/or n₂ equals 1 to 5 times administration of DNA, and wherein m₁ and/or m₂ equals 1 to 5 times administration of the viral vector. If n₁, m₁, n₂, and/or m₂ is greater than 1, the interval within steps (a), (b), (c) and/or (d) is one to twelve weeks. More particular, the interval within steps (a), (b), (c) and/or (d) is one to four weeks. In a particular embodiment, the medicament is administered at two or three weeks interval within and/or between the steps (a), (b), (c) and (d).

Specific examples are, but not limited to:
DNA—viral vector—DNA—viral vector,
DNA—DNA—viral vector—DNA—DNA—viral vector,
DNA—DNA—viral vector—DNA—viral vector,
DNA—viral vector—DNA—DNA—viral vector,
DNA—viral vector—DNA—viral vector—DNA—viral vector,
DNA—DNA—viral vector—DNA—viral vector—DNA—viral vector, and
DNA—DNA—viral vector—DNA—viral vector—viral vector.
Thus, at least two cycles of “DNA—viral vector” are present.

In a particular embodiment, the administration pattern of the medicament is as follows:

a) 2×DNA—b) viral vector—c) DNA—d) viral vector, with an interval of one to twelve weeks between the two DNA administrations in step (a), with an interval of one to twelve weeks between step (a) and (b) and (c) and (d), and with an interval of 1 week to 1 year between step (b) and (c).

More specific, the medicament is administered at one to four weeks interval between the steps (a), (b), (c), and (d). Even more specific, the medicament is administered at two or three weeks interval, plus or minus 1 to 3 days, between the steps (a), (b), (c), and (d).

In a further embodiment, the vector or composition is administered in an “effective amount”. An “effective amount” of the vector or composition is referred to as an amount required and sufficient to elicit an immune response, especially a CTL or antibody response, preferably determined subsequent to the last prime boost cycle. The “effective amount” may vary depending on the health and physical condition of the individual to be treated, the age of the individual to be treated (e.g. dosing for infants may be lower than for adults) the taxonomic group of the individual to be treated (e.g. human, non-human primate, primate, etc.), the capacity of the individual's immune system to mount an effective immune response, the degree of protection desired, the formulation of the composition, the treating doctor's assessment, the strain of the infecting pathogen and other relevant factors. The dosage of the DNA or viral vector may be administered in a single administration schedule or in a multiple administration schedule. In a multiple administration schedule, the total effective amount (or dose) is subdivided and administered at different sites, this within 24 hours, preferably within 8 hours and more preferably within 2 hours. The effective amount of the vector or composition falls in a relatively broad range that can be determined through routine trials, i.e. from 0.1 μg to 10 mg/dose and more particularly from 1 μg to 5 mg/dose for DNA; and from 1×10E4 to 5×10E10 plaque forming units (pfu)/dose for a viral vector, more particularly from 1×10E5 to 5×10E9 pfu/dose. In a specific embodiment, the effective dose of the DNA vector is 0.1 mg, 0.5 mg, 1 mg, 2 mg, 3 mg, 4 mg or 5 mg, or any value in between. In a further embodiment, the effective dose of the viral vector is 1×10E7, 2×10E7, 3×10E7, 4×10E7, 5×10E7, 1×10E8, 2×10E8, 3×10E8, 4×10E8, 5×10E8, 1×10E9, 2×10E9, 3×10E9, 4×10E9 or 5×10E9 pfu, or any value in between.

Dosage administration may be a single dose schedule or a multiple dose schedule. The vaccine may be administered in conjunction with other immunoregulatory and/or antiviral agents. The dosages, routes of administration, and dose schedules are adjusted in accordance with methodologies known in the art.

The vector of the present invention is preferably included in a composition, more specifically a pharmaceutical composition and optionally comprises a pharmaceutical acceptable excipient. The pharmaceutical composition may additionally comprise one or more further active substances and/or at least one of a pharmaceutically acceptable carrier or vehicle.

Various art-recognized delivery systems may be used to deliver the vectors of the present invention into appropriate cells. The vector can be delivered in a pharmaceutically acceptable carrier or as colloidal suspensions, or as powders, with or without diluents. They can be “naked” or associated with delivery vehicles and delivered using delivery systems known in the art. A “pharmaceutically acceptable vehicle” or “pharmaceutically acceptable carrier” includes vehicles such as water, saline, physiological salt solutions, glycerol, ethanol, etc. Auxiliary substances such as wetting or emulsifying agents, pH buffering substances, preservatives may be included in such vehicles.

Typically, the vector of the invention is prepared as an injectable, either as a liquid solution or suspension. Injection may be subcutaneous, intramuscular, intravenous, intraperitoneal, intrathecal, subcutaneous, intradermal or intraepidermal. In a preferred embodiment, the plasmid DNA is administered intramuscular and the viral vector is administered subcutaneous, intradermal or intramuscular. Other types of administration comprise implantation, suppositories, oral ingestion, enteric application, inhalation, aerosolization or nasal spray or drops. Solid forms, suitable for dissolving in, or suspension in, liquid vehicles prior to injection may also be prepared. The preparation may also be emulsified or encapsulated in liposomes which serve to target a particular tissue, such as lymphoid tissue, or to target selectively infected cells, as well as to increase the half-life of the peptide and nucleic acids composition. Liposomes include emulsions, foams, micelles, insoluble monolayers, liquid crystals, phospholipid dispersions, lamellar layers and the like.

A liquid formulation may include oils, polymers, vitamins, carbohydrates, amino acids, salts, buffers, albumin, surfactants, or bulking agents. Any physiological buffer may be used, but citrate, phosphate, succinate, and glutamate buffers or mixtures thereof are preferred.

After the liquid composition is prepared, it is preferably lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is preferably administered to subjects using those methods that are known to those skilled in the art.

The approach known as “naked DNA” is currently being used for intramuscular (IM) administration in clinical trials. To maximize the immunotherapeutic effects of DNA vaccines, an alternative method for formulating purified plasmid DNA may be desirable. A variety of methods have been described, and new techniques may become available. Cationic lipids can also be used in the formulation (see, e.g., as described by WO 93/24640; Mannino & Gould-Fogerite 1988; U.S. Pat. No. 5,279,833; WO 91/06309; and Felgner et al., 1987). In addition, glycolipids, fusogenic liposomes, peptides and compounds referred to collectively as protective, interactive, non-condensing compounds could also be complexed to purified plasmid DNA to influence variables such as stability, intramuscular dispersion, or trafficking to specific organs or cell types. Further examples of DNA-based delivery technologies include electroporation, facilitated (bupivicaine, polymers (e.g. PVP), peptide-mediated) delivery, cationic lipid complexes, particle-mediated (“gene gun”) or pressure-mediated delivery (see, e.g., U.S. Pat. No. 5,922,687), DNA formulated with charged or uncharged lipids, DNA formulated in liposomes, emulsified DNA, DNA formulated with a transfection-facilitating protein or polypeptide, DNA formulated with a targeting protein or polypeptide, DNA formulated with calcium precipitating agents, DNA coupled to an inert carrier molecule, and DNA formulated with an adjuvant. In this context it is noted that practically all considerations pertaining to the use of adjuvants in traditional vaccine formulation apply to the formulation of DNA vaccines. Detailed disclosures relating to the formulation and use of nucleic acid vaccines are available, e.g. by Donnelly J. J. et al, 1997 and 1997a.

The DNA and viral vector compositions of this invention can be provided in kit form, as a kit of parts, together with instructions for administration. Typically the kit includes the DNA and viral vector as described herein in a container, preferably in unit dosage form and preferably with instructions for administration. In a particular embodiment, the present invention provides a kit for preventing and/or treating an infection, comprising:

a) a DNA priming composition encoding an antigen derived from a pathogen; and
b) a viral vector boosting composition which directs the expression of said antigen,
wherein the viral vector is a non replicating or replication impaired recombinant poxvirus.

In a specific embodiment, the kit furthermore comprises instructions for administration, i.e. an administration pattern of at least two cycles of DNA-viral vector administration. All embodiments described herein relating to the DNA and the viral vector of the invention as well as the administration pattern are applicable to the kit comprising said compounds.

Other kit components that may also be desirable to include, for example, a sterile syringe and other desired excipients.

In a further aspect the invention provides a method for generating a T-cell response against at least one target antigen, which method comprises administering at least two doses of the priming composition, and at least two doses of the boosting composition of the kit according to the invention. Typically, the method comprises at least two cycles of DNA-viral vector administration. More specific, the present invention encompasses a method for preventing and/or treating an infection, comprising the use of DNA and a viral vector encoding an antigen derived from a pathogen wherein the administration pattern of the antigen comprises at least two cycles of DNA-viral vector administration,

wherein DNA is a plasmid DNA encoding said antigen; and
wherein the viral vector is a non replicating or replication impaired recombinant poxvirus which directs the expression of said antigen.

All embodiments described herein relating to the DNA and viral vector of the invention as well as the administration pattern are applicable to the method using said compounds.

Other arrangements of the methods and tools embodying the invention will be obvious for those skilled in the art.

It is to be understood that although preferred embodiments, specific constructions and configurations, as well as materials, have been discussed herein for the methods and tools according to the present invention, various changes or modifications in form and detail may be made without departing from the scope and spirit of this invention.

TABLE 1
HBV CTL epitopes

| HBV | Amino acid | SEQ ID |
|---|---|---|
| protein | sequence | NO |
| Pol | FLLSLGIHL | 1 |
| Pol | KYTSFPWLL | 2 |
| Env | RFSWLSLLVPF | 3 |
| Pol | FPHCLAFSYM | 4 |
| Pol | LVVDFSQFSR | 5 |
| Env | ILLLCLIFLL | 6 |
| Pol | HTLWKAGILYK | 7 |
| Env | WMMWYWGPSLY | 8 |
| Pol | YPALMPLYACI | 9 |
| Env | WLSLLVPFV | 10 |
| Env | FLLTRILTI | 11 |
| Env | IPIPSSWAF | 12 |
| Core | EYLVSFGVW | 13 |
| Core | LPSDFFPSV | 14 |
| Core | FLPSDFFPSV | 15 |
| Core | DLLDTASALY | 16 |
| Pol | SWPKFAVPNL | 17 |
| Pol | SAICSVVRR | 18 |
| Pol | LSLDVS | 19 |
| Pol | NVSIPWTHK | 20 |
| Pol | GLSRYVARL | 21 |
| Core | STLPETTVVRR | 22 |
| Pol | HPAAMPHLL | 23 |
| Env | RWMCLRRFII | 24 |
| Pol | ASFCGSPY | 25 |
| Pol | YMDDVVLGV | 26 |
| Core | LWFHISCLTF | 27 |
| Pol | TPARVTGGVF | 28 |
| Core | LTFGRETVLEY | 29 |
| Pol | QAFTFSPTYK | 30 |

Pol = polymerase
Env = envelope

TABLE 2
HBV HTL epitopes

| HBV | Amino acid | SEQ ID |
|---|---|---|
| protein | sequence | NO |
| pol | GTSFVYVPSALNPAD | 31 |
| pol | LCQVFADATPTGWGL | 32 |
| pol | RHYLHTLWKAGILYK | 33 |
| core | PHHTALRQAILCWGELMTLA | 34 |
| pol | ESRLVVDFSQFSRGN | 35 |
| pol | PFLLAQFTSAICSVV | 36 |
| env | LVPFVQWFVGLSPTV | 37 |
| pol | LHLYSHPIILGFRKI | 38 |
| pol | SSNLSWLSLDVSAAF | 39 |
| pol | LQSLTNLLSSNLSWL | 40 |
| env | AGFFLLTRILTIPQS | 41 |
| core | VSFGVWIRTPPAYRPPNAPI | 42 |
| pol | VGPLTVNEKRRLKLI | 43 |
| pol | KQCFRKLPVNRPIDW | 44 |
| pol | AANWILRGTSFVYVP | 45 |
| pol | KQAFTFSPTYKAFLC | 46 |
| PADRE | AKFVAAWTLKAAA | 47 |

Pol = polymerase
Env = envelope
PADRE = PAN DR binding peptide

The present invention is illustrated by the following Examples, which should not be understood to limit the scope of the invention to the specific embodiments therein.

### EXAMPLES

### Example 1

### Preparation of DNA and MVA Vectors

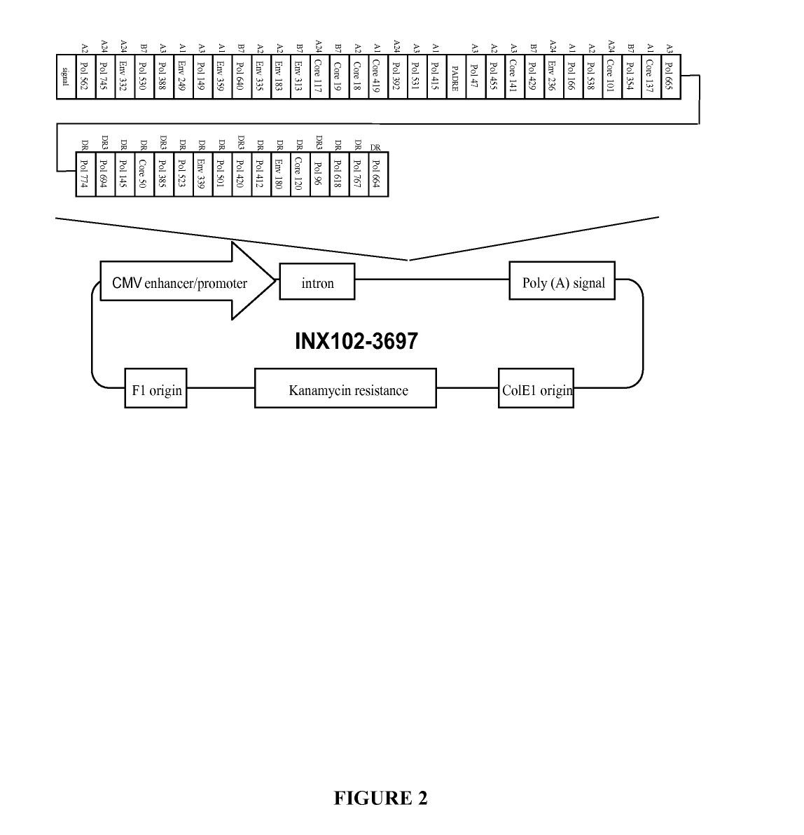

The gene construct was assembled using overlapping oligonucleotides in a PCR-based synthesis followed by subcloning into the pMB75.6 DNA plasmid vector (Valentis, Burlingame, Calif.) (Wilson C C et al., 2003). The DNA sequence was optimized to remove rare human codons and to reduce the formation of potentially deleterious secondary RNA structures. A consensus Ig kappa signal sequence was fused to the 5′ end of the gene product. Expression of the vaccine gene is driven by the CMV-IE promoter. The vaccine coding region in the expression cassette is preceded by a chimeric intron sequence and followed by the SV40 early poly-A signal (FIG. 2). Plasmid DNA (pDNA) was produced in E. coli Stb12 strain (Invitrogen, Carlsbad, Calif.) by growth at 37° C. in LB medium (Bertani G., 1951) with kanamycin (25 μg/ml) and purified using EndoFree® Plasmid Mega Kits columns according to the manufacturer's directions (Qiagen USA, Valencia, Calif.). The purified pDNA vaccine construct, designated INX102-3697, was dissolved in water. For the heterologous DNA-MVA prime-boost immunizations, the pDNA was formulated in 3.4% poly(N-vinyl pyrrolidone) (PVP; Plasdone; ISP, Wayne, N.J.), 3 mg/ml ethanol, and phosphate-buffered saline (PBS), pH 7.4 at a concentration of 2 mg/ml.

Recombinant MVA was generated by homologous recombination into deletion III of MVATGN33 (Transgene, France) using a shuttle plasmid containing the pDNA HBV vaccine gene construct functionally linked to the vaccinia virus H5R early/late promoter. The MVA construct was amplified and produced on chicken embryonic fibroblasts, purified by a multi-step low speed centrifugation process (Earl, P L. et al., 1998), and resuspended in 10 mM Tris-HCl, 5% (w/v) saccharose, 10 mM sodium glutamate, and 50 mM NaCl, pH 8.0 at an infectious titer of 2×10⁸ pfu/ml.

### Example 2

### Evaluation of Different Heterologous DNA Prime/MVA Boost Regimens in HLA-A02 Transgenic Mice

The immunogenicity of different heterologous DNA prime/MVA boost regimens was tested in F1 HLA-A02/KbxBalb/c transgenic mice. The potential of multiple heterologous DNA prime/MVA boost cycles using DNA and MVA was explored. HLA transgenic mice were immunized with one of the selected regimens. To ensure an equal distribution of mice between different groups, a randomization procedure based on gender and age (between 7 and 16 weeks) was performed.

The evaluation of the immunogenicity of HBV-derived HLA-A02-restricted epitopes and some HLA-DR restricted epitopes encoded in the polyepitope (CTL-HTL) DNA and MVA constructs was done using the protocol described herein.

### In Vivo Experimental Set-Up

The study was carried out after permission of the Local Animal Ethics Committee. In total, 5 groups of 18 F1 HLA-A02/KbxBalb/c transgenic mice were immunized (table 3).

Group 1 received 5 μg of (CTL-HTL)_HBV pvp DNA intramuscularly on week 9 and week 12, followed by a subcutaneous boost immunization with (CTL-HTL)_HBV MVA (10⁵ pfu) on week 15.

Group 2 received 5 μg of (CTL-HTL)_HBV pvp DNA intramuscularly on week 12, followed by a subcutaneous boost immunization with (CTL-HTL)_HBV MVA (10⁵ pfu) on week 15.

Group 3 received 5 μg of (CTL-HTL)_HBV pvp DNA intramuscularly on week 0 and week 3, followed by a subcutaneous boost immunization with (CTL-HTL)_HBV MVA (10⁵ pfu) on week 6. The same regimen was repeated with injection of 5 μg of (CTL-HTL)_HBV pvp DNA intramuscularly on week 9 and week 12, followed by a subcutaneous boost immunization with (CTL-HTL)_HBV MVA (10⁵ pfu) on week 15.

Group 4 received 5 μg of (CTL-HTL)_HBV pvp DNA intramuscularly on week 3 and week 6, followed by a subcutaneous boost immunization with (CTL-HTL)_HBV MVA (10⁵ pfu) on week 9. The same regimen, but using a single DNA immunization, was repeated with injection of 5 μg of (CTL-HTL)_HBV pvp DNA intramuscularly on week 12, followed by a subcutaneous boost immunization with (CTL-HTL)_HBV MVA (10⁵ pfu) on week 15.

Group 5 received 5 μg of (CTL-HTL)_HBV pvp DNA intramuscularly on week 6, followed by a subcutaneous boost immunization with (CTL-HTL)_HBV MVA (10⁵ pfu) on week 9. The same regimen was repeated with injection of 5 μg of (CTL-HTL)_{—}HBV pvp DNA intramuscularly on week 12, followed by a subcutaneous boost immunization with (CTL-HTL)_HBV MVA (10⁵ pfu) on week 15.

TABLE 3
Overview of regimens tested in different immunization groups

| Group | W0 | W3 | W6 | | W9 | | W12 | W15 | |
|---|---|---|---|---|---|---|---|---|---|
| 1 | / | / | / | | 5 | μg DNA | 5 μg DNA | 10⁵ | pfu MVA |
| 2 | / | / | / | | / | | 5 μg DNA | 10⁵ | pfu MVA |
| 3 | 5 μg DNA | 5 μg DNA | 10⁵ | pfu MVA | 5 | μg DNA | 5 μg DNA | 10⁵ | pfu MVA |
| 4 | / | 5 μg DNA | 5 | μg DNA | 10⁵ | pfu MVA | 5 μg DNA | 10⁵ | pfu MVA |
| 5 | / | / | 5 | μg DNA | 10⁵ | pfu MVA | 5 μg DNA | 10⁵ | pfu MVA |

The pvp DNA was diluted in PBS towards a concentration of 50 μg/ml and 5 μg was administered by bilateral injection of 50 μA in both m. tibialis anterior. The MVA was diluted in 10 mM Tris-HCl, 5% (w/v) saccharose, 10 mM sodium glutamate, and 50 mM NaCl, pH 8.0 towards a concentration of 10⁶ pfu/ml and 100 μl was injected subcutaneously at the base of the tail using a BD Microfine™ plus 1.0 cc insulin syringe.

### In Vitro Experimental Set-Up

Mice were euthanized and spleen cells (SPC) were isolated 12 days after the last immunization. SPC were pooled per 3 mice resulting in 6 data points per immunization group per epitope evaluated.

CD8+ cells were purified by positive magnetic bead selection on SPC using CD8a MicroBeads according to the manufacturer's protocol (Miltenyi Biotec). A direct ex vivo IFN-g ELISPOT assay was used as surrogate CTL readout. Basically, purified CD8+ cells (2×10⁵ cells/well and 5×10⁴ cells/well) were incubated with the 6 individual HBV-specific HLA-A02-restricted peptides (10 μg/mL) comprised in the CTL-HTL polyepitope construct loaded on the appropriate antigen-presenting cells (APC, Jurkat cells expressing the HLA-A2.1/Kb molecule, 2×10⁴ cells/well), in anti-mouse IFN-g antibody-coated ELISPOT plates. After 20 hours of incubation, IFN-g-producing cells were visualized by further developing the plates with biotinylated anti-mouse IFN-g antibody, streptavidin-HRP and AEC as substrate. CD4+ cells were purified by positive magnetic bead selection on SPC using CD4 MicroBeads according to the manufacturer's protocol (Miltenyi Biotec). A direct ex vivo IFN-g ELISPOT assay was used to determine the number of HBV-specific HTL type 1 CD4+ cells. Basically, purified CD4+ cells (10⁵ cells/well) were incubated with 5 individual HBV-specific HLA-DR-restricted peptides (10 μg/mL), cross-binding the murine MHC and the universal HLA-DR-restricted PADRE epitope comprised in the CTL-HTL polyepitope construct, loaded on the appropriate APC (naïve, syngeneic SPC, 2×10⁵ cells/well), in anti-mouse IFN-g antibody-coated ELISPOT plates. After 20 hours of incubation, IFN-g-producing cells were visualized by further developing the plates with biotinylated anti-mouse IFN-g antibody, streptavidin-HRP and AEC as substrate.

Basically, purified CD4+ cells (10⁵ cells/well) were incubated with 5 individual HBV-specific HLA-DR-restricted peptides (10 μg/mL) and the universal HLA-DR-restricted PADRE epitope loaded on the appropriate APC (naïve, syngeneic SPC, 2×10⁵ cells/well), in anti-mouse IL5 antibody-coated ELISPOT plates. After 48 hours of incubation, IL5-producing cells were visualized by further developing the plates with biotinylated anti-mouse IL5 antibody, streptavidin-HRP and AEC as substrate.

### Data Analysis

A response is considered positive for a specific epitope when the peptide-specific delta response is ≧30 spot forming cells/10⁶ CD8+ or CD4+ cells and when the response ratio is ≧2.

### Results

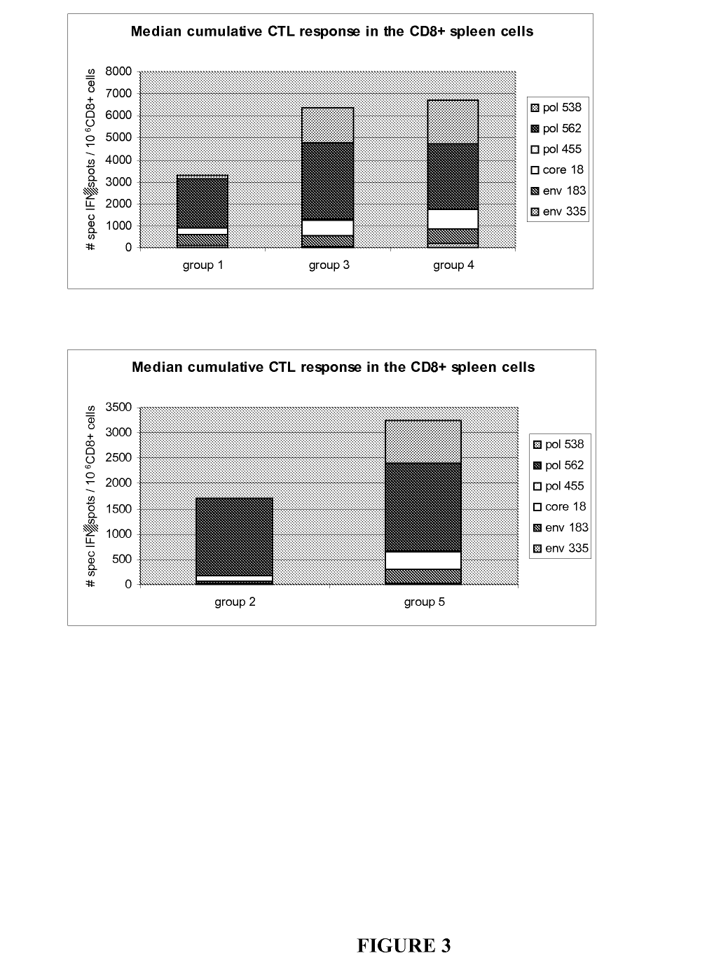

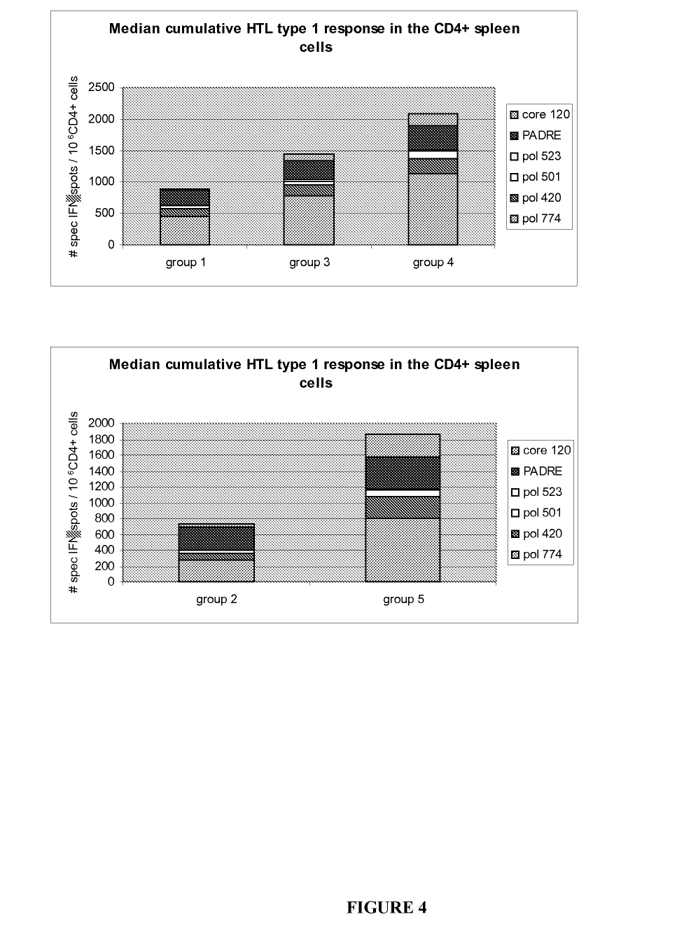

The ELISPOT results for the HBV-specific CTL responses are shown in table 4. Calculations were based on the results obtained with two different CD8+ cell densities (2×10⁵ and 5×10⁴ cells/well). Overall, responses were similar for both cell densities, indicating a good linearity of the IFN-g ELISPOT assay. When the ELISPOT reader could not count the number of spots accurately due to high responses (in the range of 300 spots) or due to too much spots around the border of the wells in the set up using 2×10⁵ cells/well, results from 5×10⁴ cells/well were used (indicated in italic in table 4), while all other responses were derived from results from 2×10⁵ cells/well. The comparison between the different regimens of the cumulative (for all 6 HLA-A02-restricted epitopes) CTL responses per immunization group is shown in FIG. 3. The ELISPOT results for the HBV-specific HTL type 1 responses are shown in table 5. The comparison between the different regimens of the cumulative (for 5 HLA-DR-restricted epitopes and PADRE) HTL type 1 responses per immunization group is shown in FIG. 4.

### CONCLUSION

From the results it is clear that stronger direct ex vivo CTL responses were elicited using a double DNA-viral vector immunization cycle (Groups 3, 4 and 5) compared to a single cycle (Groups 1-2) (p value of group 1 vs. group 3=0.0411; p value of group 1 vs. group 4=0.0152). Moreover, the double cycle regimen shows less variation between the pools of cells analysed, and more pools are positive. Increased CTL responses were elicited when a double DNA prime injection was given in the first cycle compared to a single DNA prime administration (p value of group 4 vs. group 5=0.0411).

Also the HTL type 1 responses were enhanced using the double DNA-viral vector immunization cycle.

It can thus be concluded that a double cycle DNA-viral vector immunization regimen significantly increases CTL responses and HTL type 1 responses compared to a single DNA-viral vector regimen. It is vital to use an optimal immunization schedule eliciting the most vigorous immune response since the target population, being chronically infected patients, can be immunocomprised making it more difficult to mount a sufficient immune response to clear the virus. Moreover, more pools show positive responses, and thus epitopes will show stronger immunogenicity in patients. The breadth of the immune response is of importance to minimize the risk for viral escape by targeting multiple viral epitopes simultaneously.

TABLE 4
Summary of the delta median number of HBV-specific spot forming cells/10⁶ CD8+ cells and response
ratio (2 × 10⁵ or 5 × 10⁴ cells/well, values in italic are derived from the 5 × 10⁴ cells/well condition).

| | | | Peptide used for in vitro stimulation | | | | | | | | | | | |
|---|---|---|---|---|---|---|---|---|---|---|---|---|---|---|
| | | | SEQ ID 10 | | SEQ ID 11 | | SEQ ID 15 | | SEQ ID 21 | | SEQ ID 1 | | SEQ ID 26 | |
| | | | (env335) | | (env183) | | (core18) | | (pol445) | | (pol562) | | (pol538) | |
| group | pool | immunisation | Delta | Ratio | Delta | Ratio | Delta | Ratio | Delta | Ratio | Delta | Ratio | Delta | Ratio |
| 1 | 1 | 5 μg (CTL-HTL)_HBV pvp DNA | 20 | 3.0 | 30 | 4.0 | 1000 | 101.0 | 5 | 1.5 | 1045 | 105.5 | 0 | 1.0 |
| | 2 | i.m, week 9 and week 12 | 130 | 9.7 | 820 | 55.7 | 660 | 45.0 | 95 | 7.3 | 695 | 47.3 | 185 | 13.3 |
| | 3 | (CTL-HTL)_HBV MVA (10⁵ pfu) | 89 | 90.0 | 629 | 630.0 | 19 | 20.0 | 14 | 15.0 | 4680 | 235.0 | 589 | 590.0 |
| | 4 | s.c., week 15 | 195 | 14.0 | 850 | 57.7 | 380 | 26.3 | 0 | 1.0 | 4060 | 204.0 | 75 | 6.0 |
| | 5 | | 145 | 15.5 | 370 | 38.0 | 155 | 16.5 | 15 | 2.5 | 3460 | 174.0 | 0 | 0.0 |
| | 6 | | 0 | 0.3 | 300 | 21.0 | 20 | 2.3 | 10 | 1.7 | 365 | 25.3 | 355 | 24.7 |
| | | Mean | 97 | 22.1 | 500 | 134.4 | 372 | 35.2 | 23 | 4.8 | 2384 | 131.9 | 201 | 105.8 |
| | | Median | 110 | 11.8 | 500 | 46.8 | 268 | 23.2 | 12 | 2.1 | 2253 | 139.8 | 130 | 9.7 |
| 2 | 7 | 5 μg (CTL-HTL)_HBV pvp DNA | 10 | 3.0 | 40 | 9.0 | 15 | 4.0 | 50 | 11.0 | 2500 | 126.0 | 10 | 3.0 |
| | 8 | i.m, week 12 | 25 | 3.5 | 635 | 64.5 | 1225 | 123.5 | 10 | 2.0 | 5080 | 255.0 | 170 | 18.0 |
| | 9 | (CTL-HTL)_HBV MVA (10⁵ pfu) | 290 | 20.3 | 970 | 65.7 | 665 | 45.3 | 170 | 12.3 | 2280 | 115.0 | 890 | 60.3 |
| | 10 | s.c., week 15 | 50 | 3.5 | 45 | 3.3 | 50 | 3.5 | 5 | 1.3 | 775 | 39.8 | 0 | 0.5 |
| | 11 | | 25 | 1.4 | 10 | 1.2 | 90 | 2.4 | 5 | 1.1 | 100 | 2.5 | 0 | 0.8 |
| | 12 | | 14 | 15.0 | 9 | 10.0 | 109 | 110.0 | 9 | 10.0 | 614 | 615.0 | 0 | 0.0 |
| | | Mean | 69 | 7.8 | 285 | 25.6 | 359 | 48.1 | 42 | 6.3 | 1892 | 192.2 | 178 | 13.8 |
| | | Median | 25 | 3.5 | 43 | 9.5 | 100 | 24.7 | 10 | 6.0 | 1528 | 120.5 | 5 | 1.9 |
| 3 | 13 | 5 μg (CTL-HTL)_HBV pvp DNA | 40 | 9.0 | 545 | 110.0 | 30 | 7.0 | 5 | 2.0 | 5380 | 270.0 | 2460 | 124.0 |
| | 14 | i.m, week 0 and week 3 | 80 | 9.0 | 295 | 30.5 | 1375 | 138.5 | 65 | 7.5 | 5740 | 288.0 | 545 | 55.5 |
| | 15 | (CTL-HTL)_HBV MVA (10⁵ pfu) | 15 | 4.0 | 520 | 105.0 | 515 | 104.0 | 125 | 26.0 | 2720 | 137.0 | 1680 | 85.0 |
| | 16 | s.c., week 6 | 55 | 4.7 | 1630 | 109.7 | 870 | 59.0 | 80 | 6.3 | 2240 | 113.0 | 1480 | 75.0 |
| | 17 | 5 μg (CTL-HTL)_HBV pvp DNA | −10 | 0.3 | 835 | 56.7 | 1010 | 68.3 | 10 | 1.7 | 4280 | 215.0 | 4800 | 241.0 |
| | 18 | i.m, week 9 and 12 | 0 | 1.0 | 500 | 101.0 | 300 | 61.0 | 10 | 3.0 | 2240 | 113.0 | 1365 | 274.0 |
| | | (CTL-HTL)_HBV MVA (10⁵ pfu) | | | | | | | | | | | | |
| | | s.c., week 15 | | | | | | | | | | | | |
| | | Mean | 30 | 4.7 | 721 | 85.5 | 683 | 73.0 | 49 | 7.8 | 3767 | 189.3 | 2055 | 142.4 |
| | | Median | 28 | 4.3 | 533 | 103.0 | 693 | 64.7 | 38 | 4.7 | 3500 | 176.0 | 1580 | 104.5 |
| 4 | 19 | 5 μg (CTL-HTL)_HBV pvp DNA | 195 | 40.0 | 1000 | 201.0 | 730 | 147.0 | 330 | 67.0 | 4620 | 232.0 | 2220 | 112.0 |
| | 20 | i.m, week 3 and 6 | 15 | 4.0 | 875 | 176.0 | 1085 | 218.0 | 25 | 6.0 | 3100 | 156.0 | 1705 | 342.0 |
| | 21 | (CTL-HTL)_HBV MVA (10⁵ pfu) | 44 | 45.0 | 489 | 490.0 | 574 | 575.0 | 24 | 25.0 | 2940 | 148.0 | 2360 | 119.0 |
| | 22 | s.c., week 9 | 224 | 225.0 | 209 | 210.0 | 1109 | 1110.0 | 19 | 20.0 | 3000 | 151.0 | 534 | 535.0 |
| | 23 | 5 μg (CTL-HTL)_HBV pvp DNA | 320 | 65.0 | 755 | 152.0 | 2095 | 420.0 | 475 | 96.0 | 2460 | 124.0 | 2640 | 133.0 |
| | 24 | i.m, week 12 | 470 | 2.6 | 60 | 1.2 | 125 | 1.4 | 0 | 0.8 | 2560 | 13.8 | 355 | 2.2 |
| | | (CTL-HTL)_HBV MVA (10⁵ pfu) | | | | | | | | | | | | |
| | | s.c., week 15 | | | | | | | | | | | | |
| | | Mean | 211 | 63.6 | 565 | 205.0 | 953 | 411.9 | 146 | 35.8 | 3113 | 137.5 | 1636 | 207.2 |
| | | Median | 210 | 42.5 | 622 | 188.5 | 908 | 319.0 | 25 | 22.5 | 2970 | 149.5 | 1963 | 126.0 |
| 5 | 25 | 5 μg (CTL-HTL)_HBV pvp DNA | 24 | 25.0 | 134 | 135.0 | 249 | 250.0 | 29 | 30.0 | 1069 | 1070.0 | 1054 | 1055.0 |
| | 26 | i.m, week 6 | 20 | 3.0 | 275 | 28.5 | 715 | 72.5 | 0 | 1.0 | 1700 | 86.0 | 4140 | 208.0 |
| | 27 | (CTL-HTL)_HBV MVA (10⁵ pfu) | 125 | 9.3 | 535 | 36.7 | 320 | 22.3 | 55 | 4.7 | 1760 | 89.0 | 215 | 15.3 |
| | 28 | s.c., week 9 | 0 | 1.0 | 205 | 21.5 | 805 | 81.5 | 1100 | 111.0 | 3220 | 162.0 | 410 | 42.0 |
| | 29 | 5 μg (CTL-HTL)_HBV pvp DNA | 0 | 0.9 | 310 | 5.4 | 155 | 3.2 | 0 | 0.8 | 935 | 14.4 | 1475 | 22.1 |
| | 30 | i.m, week 12 | 90 | 1.8 | 265 | 3.2 | 410 | 4.4 | 0 | 1.0 | 1740 | 88.0 | 630 | 6.3 |
| | | (CTL-HTL)_HBV MVA (10⁵ pfu) | | | | | | | | | | | | |
| | | s.c., week 15 | | | | | | | | | | | | |
| | | Mean | 43 | 6.8 | 287 | 38.4 | 442 | 72.3 | 197 | 24.7 | 1737 | 251.6 | 1321 | 224.8 |
| | | Median | 22 | 2.4 | 270 | 25.0 | 365 | 47.4 | 15 | 2.8 | 1720 | 88.5 | 842 | 32.0 |

TABLE 5
Summary of the delta median number of HBV-specific spot forming cells/10⁶ CD4+ cells and response ratio (10⁵ cells/well).

| | | | Peptide used for in vitro stimulation | | | | | | | | | | | |
|---|---|---|---|---|---|---|---|---|---|---|---|---|---|---|
| | | | SEQ ID 31 | | SEQ ID 39 | | SEQ ID 38 | | SEQ ID 36 | | SEQ ID 47 | | SEQ ID 42 | |
| | | | (pol 774) | | (pol 420) | | (pol 501) | | (pol 523) | | (PADRE) | | (core 120) | |
| group | pool | immunisation | Delta | Ratio | Delta | Ratio | Delta | Ratio | Delta | Ratio | Delta | Ratio | Delta | Ratio |
| 1 | 1 | 5 μg (CTL-HTL)_HBV pvp DNA | 230 | 12.5 | 80 | 5.0 | 10 | 1.5 | 10 | 1.5 | 150 | 8.5 | 10 | 1.5 |
| | 2 | i.m, week 9 and week 12 | 500 | 51.0 | 70 | 8.0 | 70 | 8.0 | 60 | 7.0 | 210 | 22.0 | 20 | 3.0 |
| | 3 | (CTL-HTL)_HBV MVA (10⁵ pfu) | 420 | 9.4 | 230 | 5.6 | 40 | 1.8 | 10 | 1.2 | 530 | 11.6 | 50 | 2.0 |
| | 4 | s.c., week 15 | 650 | 66.0 | 190 | 20.0 | 60 | 7.0 | 0 | 1.0 | 480 | 49.0 | 120 | 13.0 |
| | 5 | | 620 | 32.0 | 140 | 8.0 | 80 | 5.0 | 30 | 2.5 | 250 | 13.5 | 20 | 2.0 |
| | 6 | | 160 | 6.3 | 20 | 1.7 | 0 | 1.0 | 0 | 0.3 | 70 | 3.3 | 0 | 1.0 |
| | | Mean | 430 | 30 | 122 | 8 | 43 | 4 | 18 | 2 | 282 | 18 | 37 | 4 |
| | | Median | 460 | 22 | 110 | 7 | 50 | 3 | 10 | 1 | 230 | 13 | 20 | 2 |
| 2 | 7 | 5 μg (CTL-HTL)_HBV pvp DNA | 330 | 17.5 | 90 | 5.5 | 30 | 2.5 | 0 | 1.0 | 240 | 13.0 | 80 | 5.0 |
| | 8 | i.m, week 12 | 480 | 25.0 | 70 | 4.5 | 160 | 9.0 | 10 | 1.5 | 350 | 18.5 | 150 | 8.5 |
| | 9 | (CTL-HTL)_HBV MVA (10⁵ pfu) | 670 | 17.8 | 160 | 5.0 | 40 | 2.0 | 10 | 1.3 | 930 | 24.3 | 60 | 2.5 |
| | 10 | s.c., week 15 | 230 | 6.8 | 110 | 3.8 | 30 | 1.8 | 0 | 0.8 | 470 | 12.8 | 0 | 0.5 |
| | 11 | | 140 | 4.5 | 60 | 2.5 | 0 | 0.8 | 0 | 0.5 | 180 | 5.5 | 10 | 1.3 |
| | 12 | | 180 | 19.0 | 60 | 7.0 | 40 | 5.0 | 10 | 2.0 | 170 | 18.0 | 10 | 2.0 |
| | | Mean | 338 | 15 | 92 | 5 | 50 | 4 | 5 | 1 | 390 | 15 | 52 | 3 |
| | | Median | 280 | 18 | 80 | 5 | 35 | 2 | 5 | 1 | 295 | 16 | 35 | 2 |
| 3 | 13 | 5 μg (CTL-HTL)_HBV pvp DNA | 550 | 56.0 | 300 | 31.0 | 70 | 8.0 | 0 | 1.0 | 330 | 34.0 | 130 | 14.0 |
| | 14 | i.m, week 0 and week 3 | 660 | 17.5 | 140 | 4.5 | 20 | 1.5 | 50 | 2.3 | 200 | 6.0 | 110 | 3.8 |
| | 15 | (CTL-HTL)_HBV MVA (10⁵ pfu) | 899 | 900.0 | 149 | 150.0 | 59 | 60.0 | 39 | 40.0 | 389 | 390.0 | 109 | 110.0 |
| | 16 | s.c., week 6 | 890 | 45.5 | 540 | 28.0 | 50 | 3.5 | 50 | 3.5 | 270 | 14.5 | 70 | 4.5 |
| | 17 | 5 μg (CTL-HTL)_HBV pvp DNA | 1660 | 34.2 | 160 | 4.2 | 160 | 4.2 | 30 | 1.6 | 370 | 8.4 | 170 | 4.4 |
| | 18 | i.m, week 9 and 12 | 370 | 7.2 | 190 | 4.2 | 30 | 1.5 | 20 | 1.3 | 250 | 5.2 | 80 | 2.3 |
| | | (CTL-HTL)_HBV MVA (10⁵ pfu) | | | | | | | | | | | | |
| | | s.c., week 15 | | | | | | | | | | | | |
| | | Mean | 838 | 177 | 247 | 37 | 65 | 13 | 32 | 8 | 302 | 76 | 112 | 23 |
| | | Median | 775 | 40 | 175 | 16 | 55 | 4 | 35 | 2 | 300 | 11 | 110 | 4 |
| 4 | 19 | 5 μg (CTL-HTL)_HBV pvp DNA | 1350 | 34.8 | 440 | 12.0 | 130 | 4.3 | 20 | 1.5 | 930 | 24.3 | 400 | 11.0 |
| | 20 | i.m, week 3 and 6 | 1100 | 23.0 | 150 | 4.0 | 80 | 2.6 | 20 | 1.4 | 320 | 7.4 | 70 | 2.4 |
| | 21 | (CTL-HTL)_HBV MVA (10⁵ pfu) | 1280 | 26.6 | 260 | 6.2 | 110 | 3.2 | 0 | 0.8 | 450 | 10.0 | 300 | 7.0 |
| | 22 | s.c., week 9 | 550 | 12.0 | 230 | 5.6 | 30 | 1.6 | 0 | 0.8 | 420 | 9.4 | 70 | 2.4 |
| | 23 | 5 μg (CTL-HTL)_HBV pvp DNA | 860 | 44.0 | 370 | 19.5 | 100 | 6.0 | 20 | 2.0 | 310 | 16.5 | 50 | 3.5 |
| | 24 | i.m, week 12 | 1170 | 59.5 | 160 | 9.0 | 150 | 8.5 | 40 | 3.0 | 350 | 18.5 | 300 | 16.0 |
| | | (CTL-HTL)_HBV MVA (10⁵ pfu) | | | | | | | | | | | | |
| | | s.c., week 15 | | | | | | | | | | | | |
| | | Mean | 1052 | 33 | 268 | 9 | 100 | 4 | 17 | 2 | 463 | 14 | 198 | 7 |
| | | Median | 1135 | 31 | 245 | 8 | 105 | 4 | 20 | 1 | 385 | 13 | 185 | 5 |
| 5 | 25 | 5 μg (CTL-HTL)_HBV pvp DNA | 860 | 29.7 | 240 | 9.0 | 40 | 2.3 | 30 | 2.0 | 400 | 14.3 | 310 | 11.3 |
| | 26 | i.m, week 6 | 750 | 16.0 | 290 | 6.8 | 100 | 3.0 | 10 | 1.2 | 560 | 12.2 | 140 | 3.8 |
| | 27 | (CTL-HTL)_HBV MVA (10⁵ pfu) | 1040 | 18.3 | 60 | 2.0 | 70 | 2.2 | 50 | 1.8 | 400 | 7.7 | 260 | 5.3 |
| | 28 | s.c., week 9 | 1680 | 34.6 | 510 | 11.2 | 170 | 4.4 | 0 | 1.0 | 220 | 5.4 | 500 | 11.0 |
| | 29 | 5 μg (CTL-HTL)_HBV pvp DNA | 220 | 4.7 | 80 | 2.3 | 0 | 1.0 | 10 | 1.2 | 310 | 6.2 | 120 | 3.0 |
| | 30 | i.m, week 12 | 750 | 7.8 | 980 | 9.9 | 200 | 2.8 | 160 | 2.5 | 790 | 8.2 | 480 | 5.4 |
| | | (CTL-HTL)_HBV MVA (10⁵ pfu) | | | | | | | | | | | | |
| | | s.c., week 15 | | | | | | | | | | | | |
| | | Mean | 883 | 19 | 360 | 7 | 97 | 3 | 43 | 2 | 447 | 9 | 302 | 7 |
| | | Median | 805 | 17 | 265 | 8 | 85 | 3 | 20 | 2 | 400 | 8 | 285 | 5 |

### Example 3

### Evaluation of The Immunogenicity in Different HLA Transgenic Mouse Strains of a ‘Double Cycle’ DNA-MVA Regimen

The immunogenicity of a ‘double cycle’ heterologous DNA prime-MVA boost regimen in different HLA transgenic mouse strains is evaluated. Based on the results described in example 2, the selected regimen is DNA—DNA—MVA—DNA—MVA with an interval of 3 weeks between each immunization. The immunogenicity of this regimen is further explored in HLA-B07/Kb, HLA-A11/Kb, HLA-A24/Kb, and HLA-A01/Kb transgenic mice and, as a control, in F1 HLA-A02/KbxBalb/c mice. Moreover, the effect of reducing the interval between the immunizations from three to two weeks on the induced immune responses is evaluated. To ensure an equal distribution of mice between different groups, a randomization procedure based on gender and age (between 9 and 13 weeks) is performed.

The evaluation of the immunogenicity of HBV-derived HLA-restricted epitopes encoded in the polyepitope (CTL-HTL) DNA/MVA construct is done using the following protocol.

### In Vivo Experimental Set-Up

### Part 1

In total, 4 groups of 18 HLA transgenic mice (2 groups of HLA-B07/Kb mice and 2 groups of F1 HLA-A02/KbxBalb/c mice) are included (table 6).

Group 1 (HLA-B07/Kb) and group 3 (F1 HLA-A02/KbxBalb/c) receive 5 μg of (CTL-HTL)_HBV pvp DNA intramuscularly on week 0 and week 3, followed by a subcutaneous boost immunization with (CTL-HTL)_HBV MVA (10⁵ pfu) on week 6. The same regimen, but using a single DNA immunization, is repeated with injection of 5 μg of (CTL-HTL)_HBV pvp DNA intramuscularly on week 9, followed by a subcutaneous boost immunization with (CTL-HTL)_HBV MVA (10⁵ pfu) on week 12.

Group 2 (HLA-B07/Kb) and group 4 (F1 HLA-A02/KbxBalb/c) receive 5 μg of (CTL-HTL)_HBV pvp DNA intramuscularly on week 4 and week 6, followed by a subcutaneous boost immunization with (CTL-HTL)_HBV MVA (10⁵ pfu) on week 8. The same regimen, but using a single DNA immunization, is repeated with injection of 5 μg of (CTL-HTL)_HBV pvp DNA intramuscularly on week 10, followed by a subcutaneous boost immunization with (CTL-HTL)_HBV MVA (10⁵ pfu) on week 12.

TABLE 6
Overview of regimens tested in different immunization groups

| Group | strain | W0 | W3 | W4 | W6 | W8 | W9 | W10 | W12 |
|---|---|---|---|---|---|---|---|---|---|
| 1 | HLA-B07/K^{b} | DNA | DNA | / | MVA | / | DNA | / | MVA |
| 2 | HLA-B07/K^{b} | / | / | DNA | DNA | MVA | / | DNA | MVA |
| 3 | F1 HLA-A02/K^{b} | DNA | DNA | / | MVA | / | DNA | / | MVA |
| 4 | F1 HLA-A02/K^{b} | / | / | DNA | DNA | MVA | / | DNA | MVA |

The pvp DNA is diluted in PBS towards a concentration of 50 μg/ml and 5 μg is administered by bilateral injection of 50 μl in both m. tibialis anterior.

The MVA is diluted in 10 mM Tris-HCl, 5% (w/v) saccharose, 10 mM sodium glutamate, and 50 mM NaCl, pH 8.0 toward a concentration of 10⁶ pfu/ml and 100 μl is injected subcutaneously at the base of the tail using a BD Microfine™ plus 1.0 cc insulin syringe.

### Part 2

In total, 4 groups of 18 HLA transgenic mice (2 groups of HLA-A11/Kb mice and 2 groups of HLA-A24/Kb mice) are included (table 7).

Group 1 (HLA-A11/Kb) and group 3 (HLA-A24/Kb mice) receive 5 μg of (CTL-HTL)_HBV pvp DNA intramuscularly on week 0 and week 3, followed by a subcutaneous boost immunization with (CTL-HTL)_HBV MVA (10⁵ pfu) on week 6. The same regimen, but using a single DNA immunization, is repeated with injection of 5 μg of (CTL-HTL)_HBV pvp DNA intramuscularly on week 9, followed by a subcutaneous boost immunization with (CTL-HTL)_HBV MVA (10⁵ pfu) on week 12.

Group 2 (HLA-A11/Kb) and group 4 (HLA-A24/Kb mice) receive 5 μg of (CTL-HTL)_HBV pvp DNA intramuscularly on week 4 and week 6, followed by a subcutaneous boost immunization with (CTL-HTL)_HBV MVA (10⁵ pfu) on week 8. The same regimen, but using a single DNA immunization, is repeated with injection of 5 μg of (CTL-HTL)_HBV pvp DNA intramuscularly on week 10, followed by a subcutaneous boost immunization with (CTL-HTL)_HBV MVA (10⁵ pfu) on week 12.

TABLE 7
Overview of regimens tested in different immunization groups

| Group | strain | W0 | W3 | W4 | W6 | W8 | W9 | W10 | W12 |
|---|---|---|---|---|---|---|---|---|---|
| 1 | HLA-A11/K^{b} | DNA | DNA | / | MVA | / | DNA | / | MVA |
| 2 | HLA-A11/K^{b} | / | / | DNA | DNA | MVA | / | DNA | MVA |
| 3 | HLA-A24/K^{b} | DNA | DNA | / | MVA | / | DNA | / | MVA |
| 4 | HLA-A24/K^{b} | / | / | DNA | DNA | MVA | / | DNA | MVA |

Dilutions and injections of the different products is performed as described as above (part 1).

### Part 3

In total, 2 groups of 18 HLA-A01/Kb transgenic mice are included (table 8). Group 1 receives 5 μg of (CTL-HTL)_HBV pvp DNA intramuscularly on week 0 and week 3, followed by a subcutaneous boost immunization with (CTL-HTL)_HBV MVA (10⁵ pfu) on week 6. The same regimen, but using a single DNA immunization, is repeated with injection of 5 μg of (CTL-HTL)_HBV pvp DNA intramuscularly on week 9, followed by a subcutaneous boost immunization with (CTL-HTL)_HBV MVA (10⁵ pfu) on week 12.

Group 2 receives 5 μg of (CTL-HTL)_HBV pvp DNA intramuscularly on week 4 and week 6, followed by a subcutaneous boost immunization with (CTL-HTL)_HBV MVA (10⁵ pfu) on week 8. The same regimen, but using a single DNA immunization, is repeated with injection of 5 μg of (CTL-HTL)_HBV pvp DNA intramuscularly on week 10, followed by a subcutaneous boost immunization with (CTL-HTL)_HBV MVA (10⁵ pfu) on week 12.

TABLE 8
Overview of regimens tested in different immunization groups

| Group | strain | W0 | W3 | W4 | W6 | W8 | W9 | W10 | W12 |
|---|---|---|---|---|---|---|---|---|---|
| 1 | HLA-A01/K^{b} | DNA | DNA | / | MVA | / | DNA | / | MVA |
| 2 | HLA-A01/K^{b} | / | / | DNA | DNA | MVA | / | DNA | MVA |

Dilutions and injections of the different products are performed as described as above (part 1).

### In Vitro Experimental Set-Up

Mice are euthanized and spleen cells (SPC) are isolated 12 days after immunization. SPC are pooled per 3 mice resulting in 6 data points per immunization group per epitope evaluated.

CD8+ cells are purified by positive magnetic bead selection on SPC using CD8a MicroBeads according to the manufacturer's protocol (Miltenyi Biotec). A direct ex vivo IFN-g ELISPOT assay is used as surrogate CTL readout. Basically, purified CD8+ cells (2×10⁵ and 5×10⁴ cells/well) are incubated with the individual HBV-specific HLA-restricted peptides (10 μg/mL) loaded on the appropriate antigen-presenting cells (APC), in anti-mouse IFN-g antibody-coated ELISPOT plates. After 20 hours of incubation, IFN-g producing cells are visualized by further developing the plates with biotinylated anti-mouse IFN-g antibody, streptavidin-HRP and AEC as substrate. APC used for different HLA-restricted peptides are Jurkat cells expressing the HLA-B07/Kb molecule (2×10⁴ cells/well) for HLA-B07, Jurkat cells expressing the HLA-A20.1 Kb molecule (2×10⁴ cells/well) for HLA-A02, naïve spleen cells from non-immunized HLA-A11/Kb mice (2×10⁵ cells/well) for HLA-A11, LCL721.221 cells expressing the HLA-A24/Kb molecule (10⁴ cells/well) for HLA-A24, and naïve spleen cells from non-immunized HLA-A01/Kb mice (2×10⁵ cells/well) for HLA-A01.

Only for F1 HLA-A02/KbxBalb/c transgenic mice, CD4+ cells are purified by positive magnetic bead selection on SPC using CD4 MicroBeads according to the manufacturer's protocol (Miltenyi Biotech).

A direct ex vivo IFN-g ELISPOT assay is used to determine the number of HBV-specific HTL type 1 CD4+ cells. Basically, purified CD4+ cells (10⁵ cells/well) are incubated with 5 individual HBV-specific HLA-DR-restricted peptides (10 μg/mL) and the universal HLA-DR-restricted PADRE epitope loaded on the appropriate APC (naïve, syngeneic SPC, 2×10⁵ cells/well), in anti-mouse IFN-g antibody-coated ELISPOT plates. After 20 hours of incubation, IFN-g producing cells are visualized by further developing the plates with biotinylated anti-mouse IFN-g antibody, streptavidin-HRP and AEC as substrate.

A direct ex vivo IL5 ELISPOT assay is used to determine the number of HBV-specific HTL-type 2 CD4+ cells. Basically, purified CD4+ cells (10⁵ cells/well) are incubated with 5 individual HBV-specific HLA-DR-restricted peptides (10 μg/mL) and the universal HLA-DR-restricted PADRE epitope loaded on the appropriate APC (naïve, syngeneic SPC, 2×10⁵ cells/well), in anti-mouse IL5 antibody-coated ELISPOT plates. After 48 hours of incubation, IL5 producing cells are visualized by further developing the plates with biotinylated anti-mouse IL5 antibody, streptavidin-HRP and AEC as substrate.

### Data Analysis

A response is considered positive for a specific epitope when the peptide-specific delta response is ≧30 spot forming cells/10⁶ CD8+ or CD4+ cells and when the response ratio is ≧2.

### Example 4

### Repeated Dose Toxicity Study

The potential toxicity and local tolerance of a ‘double cycle’ heterologous DNA prime-MVA boost regimen is evaluated in 48 New Zealand White rabbits (24 males and 24 females, 2 to 4 months old, between 2.0 kg and 3.5 kg). On completion of the immunization period designated animals are held for a 4- or 14-day period, in order to evaluate reversibility of any findings.

### In Vivo Experimental Set-Up

The study is carried out after review and approval by the Ethical Committee and the Biosafety Committee.

In total, 2 groups of 24 rabbits (12 males and 12 females) are included.

Group 1 receives a saline solution intramuscularly on week 0 and week 3, followed by a subcutaneous boost immunization with a saline solution on week 6. The same regimen is repeated with injection of a saline solution intramuscularly on week 9, followed by a subcutaneous boost immunization with a saline solution on week 12.

Group 2 receives 4 mg of (CTL-HTL)_HBV pvp DNA intramuscularly on week 0 and week 3, followed by a subcutaneous boost immunization with (CTL-HTL)_HBV MVA (2×10⁸ pfu) on week 6. The same regimen, but using a single DNA immunization, is repeated with injection of 4 mg of (CTL-HTL)_HBV pvp DNA intramuscularly on week 9, followed by a subcutaneous boost immunization with (CTL-HTL)_HBV MVA (2×10⁸ pfu) on week 12.

Four days after the last injection, the six first surviving animals/sex/group are sacrificed and the remaining animals are kept for another 14 days (recovery period).

### Clinical Examinations, Laboratory Investigations and Pathology

At multiple time points, each animal is checked for morbidity, mortality, clinical signs, local tolerance, body weight, body temperature, and ophtalmological abnormalities.

Also haematology and blood chemistry parameters are determined for all animals before and after the immunization period.

Four or fourteen days after the last immunization a pathological examination is performed.

### Results

The treatment was clinically well tolerated, locally and systemically, and did not induce any irreversible changes at hematology or blood biochemistry. Specifically, moderate local reactions (erythema and edema) were seen after administration of (CTL-HTL)_HBV MVA. Microscopically, minimal to marked subcutis inflammatory cells, collagen degradation and degenerative/necrotic myopathy of the subcutaneous muscle were noted in the test item-treated male and female rabbits. This was associated with subcutis hemorrhage in occasional individuals. These findings were partially reversible.

Slight erythemas were seen after administration of (CTL-HTL)_HBV pvp DNA. At necropsy and microscopy, no relevant findings were noted at the injection sites at the end of the treatment or treatment-free period in control or treated animals. Higher fibrinogen concentrations were noted in treated males and females at the end of the treatment period, but these had returned to pre-dose values at the end of the treatment-free period and were therefore considered to almost reversible with time. The albumin/globulin ratio was significantly lower in treated animals in comparison to controls at the end of the treatment period and was considered to be only partially reversible at the end of the treatment-free period as the female values remained low.

Consequently, under the conditions of this study, it can be concluded that the test items, (CTL-HTL)_HBV pvp DNA and (CTL-HTL)_HBV MVA, when administered by the intramuscular and subcutaneous routes, respectively, were well tolerated at the injection sites and did not result in any evidence of systemic toxicity.

### Example 5

### Phase I Study

The safety and tolerability, as well as the immunogenicity of the ‘double cycle’ heterologous DNA prime-MVA boost regimen as given in Example 4 is evaluated in 12 to 24 subjects.

### Study Design

(CTL-HTL)_HBV pvp DNA is administered intramuscularly at weeks 0, 3, and 9: 1 mL is injected in one deltoid muscle and 1 mL is injected in the contralateral deltoid muscle. At every time point a total of 4 mg is administered.

(CTL-HTL)_HBV MVA is administered subcutaneously at weeks 6 and 12: 0.5 mL is injected in one arm and 0.5 mL is injected in the contralateral arm. At every time point, a total of 2×10⁸ pfu is administered.

After the last immunization, a follow-up period to evaluate long-term safety and immunogenicity is included.

### Safety Evaluation

Hematology, blood chemistry, HBV serology, urine, vital signs, physical conditions, and body weight are regularly monitored.

### Immunogenicity Evaluation

IFN-g ELISPOT assays are performed to measure HBV CTL peptide-specific CD8+ T-cell responses in peripheral blood mononuclear cells (PBMC) obtained at multiple time points.

Proliferation assays are performed to measure HBV HTL peptide-specific CD4+ T-cell responses in PBMC obtained at multiple time points.

### REFERENCES

Alexander, J. et al., J. Immunol. 159:4753, 1997
Alexander J. et al., Hum Immunol 64(2): 211-223, 2003
Ausubel et al, Current Protocols in Molecular Biology (1994)
Bertani, G., J. Bacteriol. Bd. 62, Nr. 3, S. 293-300, 1951
Bertoni, R. et al., J. Clin. Invest. 100:503, 1997
Celis, E. et al., Proc. Natl. Acad. Sci. USA 91:2105, 1994
Diepolder, H. M. et al., J. Virol. 71:6011, 1997
Donnelly J J, Ulmer J B, Shiver J W, Liu M A. DNA vaccines. Annu Rev Immunol. 1997; 15:617-48.
Donnelly J J, Ulmer J B, Liu M A. DNA vaccines. Life Sci. 1997a; 60(3):163-72.
Doolan, D. L. et al., Immunity 7:97, 1997a
Doolan, D. L. et al., Immunity 7:97-112, 1997
Earl P L et al., (1998) in Current Protocols in Molecular Biology, eds. Ausubel et al. (Greene & Wiley, New York), Vol. 2, pp. 16.17.1-16.17.19.
Felgner, et al., Proc. Nat'l Acad. Sci. USA 84:7413, 1987
Ishioka et al., J Immunol, Vol. 162(7):3915-25 (1999).
Kawashima, 1. et al., Human Immunol. 59:1, 1998
Mannino & Gould-Fogerite, BioTechniques 6(7): 682, 1988
Mateo et al., J Immunol, Vol. 163(7):4058-63 (1999).
McConkey S J, et al. Nat. Med. 2003 June; 9(6):729-35.
Mwau M, et al. J Gen Virol. 2004 April; 85(Pt 4):911-9.
Rehermann, B. et al., J. Exp. Med. 181:1047, 1995
Sambrook et al., Molecular cloning, A Laboratory Manual (2^{nd} ed. 1989)
Sette, et al, J Immunol 153:5586-5592, 1994
Sette, et al., Mol. Immunol. 31: 813 (1994).
Threlkeld, S. C. et al., J. Immunol. 159:1648, 1997
Thomson et al., J Immunol, Vol. 160(4):1717-23 (1998).
Tsai S L, Huang S N. J Gastroenterol Hepatol. 1997 October; 12(9-10):S227-35.
Tsai, V. et al., J. Immunol. 158:1796, 1997
Vuola J M, et al. J. Immunol. 2005 Jan. 1; 174(1):449-55.
Wentworth, P. A. et al., Mol. Immunol. 32:603, 1995
Wentworth, P. A. et al., J. Immunol. 26:97, 1996
Wilson C. C., et al., J. Immunol. 171:5611-5623 (2003)
Woodberry et al., J Virol, Vol. 73(7):5320-5 (1999).

1. A method for preventing and/or treating an infection comprising administering DNA and a viral vector encoding an antigen derived from a pathogen,
wherein the administration pattern comprises at least two cycles of DNA-viral vector administration,
wherein DNA is a plasmid DNA encoding said an antigen, and
wherein the viral vector is a non replicating or replication impaired recombinant poxvirus which directs the expression of said antigen.

2. The method according to claim 1, with an interval of one to twelve weeks between the DNA and viral vector administration and 1 week to 1 year between two cycles.

3. The method according to claim 1, wherein the DNA is administered intramuscular, and wherein the viral vector is administered subcutaneous, intradermal or intramuscular.

4. The method according to claim 1, wherein the dose of DNA is between 1 μg and 5 mg and the dose of the viral vector is between 1×10E5 and 5×10E9 pfu.

5. The method according to claim 1, wherein the administration pattern of the medicament comprises at least the following:
a) n₁ DNA—b) m₁ viral vector—c) n₂ DNA—d) m₂ viral vector, wherein n₁ and/or n₂ equals 1 to 5 times administration of DNA, and wherein m₁ and/or m₂ equals 1 to 5 times administration of the viral vector.

6. The method according to claim 5, wherein the interval within steps (a), (b), (c) and/or (d) is one to twelve weeks if n₁, m₁ n₂, and/or m₂ is greater than 1.

7. The method according to claim 6, wherein the medicament is administered at one to four weeks interval within and/or between the steps (a), (b), (c) and (d).

8. The method according to claim 1, wherein the non replicating or replication impaired recombinant poxvirus is a vaccinia virus.

9. The method according to claim 8, wherein the vaccinia virus is MVA.

10. The method according to claim 1, wherein the pathogen is a virus, wherein the antigen is a viral antigen, and/or wherein the infection is a viral infection.

11. (canceled)

12. The method according to claim 10, wherein the viral antigen is obtained from HBV, HCV, HIV or HPV, and/or wherein the viral infection is a HCV, HBV, HIV, or HPV infection.

13. (canceled)

14. (canceled)

15. The method according to claim 1, wherein the antigen is a polyepitope construct.

16. The method according to claim 15, wherein the polyepitope construct comprises at least 5 CTL epitopes or wherein the polyepitope construct comprises at least two of the CTL epitopes selected from the group consisting of SEQ ID NO 1-30.

17. (canceled)

18. The method according to claim 16, wherein the polyepitope construct further comprises at least one HTL epitope.

19. The method according to claim 18, wherein at least one HTL epitope is selected from the group consisting of: SEQ ID NO 31-47.

20. The method according to claim 15, wherein the poly epitope construct comprises the following CTL epitopes: SEQ ID NO 1-30.

21. The method according to claim 18, wherein the polyepitope construct further comprises the following HTL epitopes: SEQ ID NO 31-47.

22. The method according to claim 1, wherein the administration pattern is:
DNA—3 weeks—DNA—3 weeks—viral vector—3 weeks—DNA—3 weeks—viral vector, whereby
the DNA dosage is 4 mg for intramuscular injection; and
the viral vector dosage is 2×10E8 pfu for subcutaneous injection.

23. The method according to claim 3, wherein the DNA is administered via electroporation, via facilitated delivery, via cationic lipid complexes, via particle-mediated or via pressure-mediated delivery.

24. A method for preventing and/or treating an infection comprising at least two cycles of DNA—viral vector administration, wherein DNA is a plasmid DNA encoding an antigen derived from a pathogen, and wherein the viral vector is a non replicating or replication impaired recombinant poxvirus which directs the expression of said antigen.

25. Medicament comprising
a) a DNA priming composition encoding an antigen derived from a pathogen; and
b) a viral vector boosting composition which directs the expression of said antigen, wherein the viral vector is a non replicating or replication impaired recombinant poxvirus,
for use in preventing and/or treating an infection by at least two cycles of DNA-viral vector administration.

26. A kit for preventing and/or treating an infection, comprising:
a) a DNA priming composition encoding an antigen derived from a pathogen; and
b) a viral vector boosting composition which directs the expression of said antigen, wherein the viral vector is a non replicating or replication impaired recombinant poxvirus.

27. A kit according to claim 26, further comprising instructions for administration comprising an administration pattern of at least two cycles of DNA—viral vector.


SEQUENCE LISTING (49 sequences)

SEQ ID NO: 1 - protein, 9 aa, Hepatitis B Virus
FLLSLGIHL

SEQ ID NO: 2 - protein, 9 aa, Hepatitis B Virus
KYTSFPWLL

SEQ ID NO: 3 - protein, 11 aa, Hepatitis B Virus
RFSWLSLLVPF

SEQ ID NO: 4 - protein, 10 aa, Hepatitis B Virus
FPHCLAFSYM

SEQ ID NO: 5 - protein, 10 aa, Hepatitis B Virus
LVVDFSQFSR

SEQ ID NO: 6 - protein, 10 aa, Hepatitis B Virus
ILLLCLIFLL

SEQ ID NO: 7 - protein, 11 aa, Hepatitis B Virus
HTLWKAGILYK

SEQ ID NO: 8 - protein, 11 aa, Hepatitis B Virus
WMMWYWGPSLY

SEQ ID NO: 9 - protein, 11 aa, Hepatitis B Virus
YPALMPLYACI

SEQ ID NO: 10 - protein, 9 aa, Hepatitis B Virus
WLSLLVPFV

SEQ ID NO: 11 - protein, 9 aa, Hepatitis B Virus
FLLTRILTI

SEQ ID NO: 12 - protein, 9 aa, Hepatitis B Virus
IPIPSSWAF

SEQ ID NO: 13 - protein, 9 aa, Hepatitis B Virus
EYLVSFGVW

SEQ ID NO: 14 - protein, 9 aa, Hepatitis B Virus
LPSDFFPSV

SEQ ID NO: 15 - protein, 10 aa, Hepatitis B Virus
FLPSDFFPSV

SEQ ID NO: 16 - protein, 10 aa, Hepatitis B Virus
DLLDTASALY

SEQ ID NO: 17 - protein, 10 aa, Hepatitis B Virus
SWPKFAVPNL

SEQ ID NO: 18 - protein, 9 aa, Hepatitis B Virus
SAICSVVRR

SEQ ID NO: 19 - protein, 6 aa, Hepatitis B Virus
LSLDVS

SEQ ID NO: 20 - protein, 9 aa, Hepatitis B Virus
NVSIPWTHK

SEQ ID NO: 21 - protein, 9 aa, Hepatitis B Virus
GLSRYVARL

SEQ ID NO: 22 - protein, 11 aa, Hepatitis B Virus
STLPETTVVRR

SEQ ID NO: 23 - protein, 9 aa, Hepatitis B Virus
HPAAMPHLL

SEQ ID NO: 24 - protein, 10 aa, Hepatitis B Virus
RWMCLRRFII

SEQ ID NO: 25 - protein, 8 aa, Hepatitis B Virus
ASFCGSPY

SEQ ID NO: 26 - protein, 9 aa, Hepatitis B Virus
YMDDVVLGV

SEQ ID NO: 27 - protein, 10 aa, Hepatitis B Virus
LWFHISCLTF

SEQ ID NO: 28 - protein, 10 aa, Hepatitis B Virus
TPARVTGGVF

SEQ ID NO: 29 - protein, 11 aa, Hepatitis B Virus
LTFGRETVLEY

SEQ ID NO: 30 - protein, 10 aa, Hepatitis B Virus
QAFTFSPTYK

SEQ ID NO: 31 - protein, 15 aa, Hepatitis B Virus
GTSFVYVPSALNPAD

SEQ ID NO: 32 - protein, 15 aa, Hepatitis B Virus
LCQVFADATPTGWGL

SEQ ID NO: 33 - protein, 15 aa, Hepatitis B Virus
RHYLHTLWKAGILYK

SEQ ID NO: 34 - protein, 20 aa, Hepatitis B Virus
PHHTALRQAILCWGELMTLA

SEQ ID NO: 35 - protein, 15 aa, Hepatitis B Virus
ESRLVVDFSQFSRGN

SEQ ID NO: 36 - protein, 15 aa, Hepatitis B Virus
PFLLAQFTSAICSVV

SEQ ID NO: 37 - protein, 15 aa, Hepatitis B Virus
LVPFVQWFVGLSPTV

SEQ ID NO: 38 - protein, 15 aa, Hepatitis B Virus
LHLYSHPIILGFRKI

SEQ ID NO: 39 - protein, 15 aa, Hepatitis B Virus
SSNLSWLSLDVSAAF

SEQ ID NO: 40 - protein, 15 aa, Hepatitis B Virus
LQSLTNLLSSNLSWL

SEQ ID NO: 41 - protein, 15 aa, Hepatitis B Virus
AGFFLLTRILTIPQS

SEQ ID NO: 42 - protein, 20 aa, Hepatitis B Virus
VSFGVWIRTPPAYRPPNAPI

SEQ ID NO: 43 - protein, 15 aa, Hepatitis B Virus
VGPLTVNEKRRLKLI

SEQ ID NO: 44 - protein, 15 aa, Hepatitis B Virus
KQCFRKLPVNRPIDW

SEQ ID NO: 45 - protein, 15 aa, Hepatitis B Virus
AANWILRGTSFVYVP

SEQ ID NO: 46 - protein, 15 aa, Hepatitis B Virus
KQAFTFSPTYKAFLC

SEQ ID NO: 47 - protein, 13 aa, Hepatitis B Virus
AKFVAAWTLKAAA

SEQ ID NO: 48 - protein, 5 aa, Artificial sequence
  Synthetic
GPGPG

SEQ ID NO: 49 - protein, 744 aa, Artificial Sequence
  Synthetic
MGMQVQIQSLFLLLLWVPGSRGFLLSLGIHLNAAAKYTSFPWLLNAAARFSWLSLLVPFNAAFPHCLAFSYMKAALVVDFSQFSRGAILLLCLIFLLNAAAHTLWKAGILYKKAWMMWYWGPSLYKAYPALMPLYACIGAAAWLSLLVPFVNAAAGFLLTRILTINAAAIPIPSSWAFKAAAEYLVSFGVWNLPSDFFPSVKAAAFLPSDFFPSVKAAADLLDTASALYNSWPKFAVPNLKAAASAICSVVRRKLSLDVSAAFYNAAAKFVAAWTLKAAAKAANVSIPWTHKGAAGLSRYVARLNAAASTLPETTVVRRKHPAAMPHLLKAAARWMCLRRFIINASFCGSPYKAAYMDDVVLGVNALWFHISCLTFKAAATPARVTGGVFKAAALTFGRETVLEYKQAFTFSPTYKNAGTSFVYVPSALNPADGPGPGLCQVFADATPTGWGLGPGPGRHYLHTLWKAGILYKGPGPGPHHTALRQAILCWGELMTLAGPGPGESRLVVDFSQFSRGNGPGPGPFLLAQFTSAICSVVGPGPGLVPFVQWFVGLSPTVGPGPGLHLYSHPIILGFRKIGPGPGSSNLSWLSLDVSAAFGPGPGLQSLTNLLSSNLSWLGPGPGAGFFLLTRILTIPQSGPGPGVSFGVWIRTPPAYRPPNAPIGPGPGVGPLTVNEKRRLKLIGPGPGKQCFRKLPVNRPIDWGPGPGAANWILRGTSFVYVPGPGPGKQAFTFSPTYKAFLCG